(19) United States
(12) Patent Application Publication
(10) Pub. No.: US 2014/0234233 A1
(43) Pub. Date: Aug. 21, 2014

(54) Associative Thickeners For Aqueous Preparations

(71) Applicants: Holger Türk, Mannheim (DE); Volker Wendel, Seeheim-Jugenheim (DE); Anna Cristadoro, Heppenheim (DE); Rabie Al-Hellani, Ludwigshafen (DE); Bernd Bruchmann, Freinsheim (DE)

(72) Inventors: Holger Türk, Mannheim (DE); Volker Wendel, Seeheim-Jugenheim (DE); Anna Cristadoro, Heppenheim (DE); Rabie Al-Hellani, Ludwigshafen (DE); Bernd Bruchmann, Freinsheim (DE)

(73) Assignee: BASF SE, Ludwigshafen (DE)

(21) Appl. No.: 14/263,083

(22) Filed: Apr. 28, 2014

Related U.S. Application Data
(63) Continuation of application No. 13/222,669, filed on Aug. 31, 2011.
(60) Provisional application No. 61/378,948, filed on Sep. 1, 2010.

(51) Int. Cl. C07C 43/10; A61Q 17/04; A61Q 5/06; A61Q 1/00; A61Q 1/10; A61Q 19/00; A61Q 9/02; A61Q 11/00; A61Q 19/04; A61Q 5/12; A61Q 15/00; C08G 18/48; A61L 24/04; A61Q 19/10; A61Q 5/02; A61K 8/87

(52) U.S. Cl. CPC C07C 43/10; A61K 8/87; A61Q 17/04; A61Q 5/06; A61Q 1/00; A61Q 1/10; A61Q 19/007; A61Q 9/02; A61Q 19/002; A61Q 11/00; A61Q 19/04; A61Q 5/12; A61Q 15/00; C08G 18/4833; A61L 24/046; A61Q 19/10; A61Q 5/02
USPC 424/54; 568/613; 514/772.3; 424/70.17; 424/65; 528/76; 524/591; 510/405

(57) ABSTRACT

Described are associative thickeners based on compounds which comprise at least one hydrophobically modified polyether dendron end group.

### CROSS-REFERENCE TO RELATED APPLICATIONS

This application is a continuation and claims the benefit of priority of U.S. application Ser. No. 13/222,669, filed Aug. 31, 2011, which claims the benefit of priority under 35 U.S.C. §119(e) to U.S. Provisional Application No. 61/378,948, filed Sep. 1, 2010, the disclosures of which are incorporated herein by reference in their entireties.

### BACKGROUND

In addition to pseudoplastic thickeners for aqueous systems such as cellulose ethers and acrylate thickeners, an important part is also played by thickeners whose effect is associative. In terms of their structural makeup, the associative thickeners are compounds having decidedly hydrophilic and hydrophobic segments, which on account of their segmented structure are capable of forming more or less stable associations with themselves and/or with the coating constituents (binders, pigments, fillers, etc.). By variation in the chemical structure it is possible in principle to vary the stability of the associations and hence the rheological properties of a coating system.

The best-known associative thickeners are polyurethane-based polymers, referred to as HEUR thickeners (“nonionic hydrophobically modified ethylene oxide urethane block copolymer”).

The HEUR-type associative thickeners, described as long ago as the end of the 1970s in U.S. Pat. No. 4,079,028 (Rohm & Haas), are constructed from linear and/or branched polyethylene glycol blocks and hydrophobic segments, which are linked to one another generally via urethane groups.

The thickening effect of the usual HEUR thickeners derives from the fact that the hydrophilic polyethylene glycol sections secure compatibility with water, and the hydrophobic sections, via association, build up a three-dimensional molecular assembly that provides viscosity.

EP 761780 and EP 1111014 (Asahi Denka) describe polyurethane thickeners constructed of polyethylene glycols as internal hydrophilic units, of linear diisocyanates as inner hydrophobic units, and of ethoxylated, alkyl-chain-branched alcohols as hydrophilic-hydrophobic chain ends.

Associative thickeners are used in numerous fields, including, increasingly, that of cosmetology.

EP 1584331 and EP 1013264 (Shiseido) describe substantially the use of the polyurethanes described in aforementioned EP 761780 as thickeners for cosmetic preparations.

WO 2006/002813 (Cognis) describes polyurethane thickeners for various applications in aqueous media. The thickeners comprise hydrophilic polyols having at least two hydroxyl groups, one or more hydrophobic compounds such as long-chain alcohols, for example, and at least difunctional isocyanates. An excess of NCO groups produces branches within the chain. These polyurethanes are used, for example, in cosmetic preparations.

WO 02/88212 (Cognis) describes polyurethanes formed from ethoxylated long-chain alcohols and cyclic diisocyanate oligomers, isocyanurates, for example, and describes their use in cosmetology.

EP 725097 (Bayer) describes polyurethane thickeners prepared by reacting polyethers, produced by alkoxylation of alcohols or alkylphenols, with polyisocyanates, the ratio of NCO to OH equivalents is in the range from 0.9:1 to 1.2:1. These thickeners are proposed, for example, for use in aqueous emulsion paints.

WO 2009/135856 and WO 2009/135857 describe polyurethanes which can be dispersed in water and which have a substantially linear backbone composed of alternating hydrophilic and hydrophobic sections, and describe the uses of these polyurethanes, in cosmetology, for example.

DE 102008030992 relates to linear-dendritic polyglycerol compounds, to processes for preparing them, and to their use for solubilizing hydrophobic substances, more particularly as vehicles or transport systems for active compounds and/or signaling compounds.

### SUMMARY

According to one or more embodiments, provided are associative thickeners based on compounds which comprise at least one hydrophobically modified polyether dendron end group, as well as aqueous preparations comprising at least one of these associative thickeners.

### DETAILED DESCRIPTION

In one or more embodiments, provided are thickeners that are suitable for cosmetic preparations and have defined properties. Examples of the desired properties of such thickeners include the following:
particularly efficient and stable thickening effect even with high levels of electrolytes and other ingredients in the preparations, such as pigments, for example;
stable thickening effect up to temperatures well above room temperature—for example, up to 40° C.;
stable thickening effect over an extremely broad pH range, as for example in the range from pH 2 to pH 13;
compatibility with customary ingredients of cosmetic preparations;
ease of incorporation into cosmetic preparations;
substantial absence of cosmetically objectionable ingredients such as, for example, toxic heavy metals.

One or more embodiments provide compounds having end groups based on polyether dendrons.

According to one or more embodiments, provided are compounds comprising at least one group of the general formula (I)

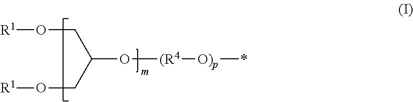

where the definitions are as follows:
R¹: independently at each occurrence C₄-C₄₀-alkyl, C₃-C₁₀-cycloalkyl, C₆-C₃₀-aryl, C₇-C₄₀-aralkyl, C₇-C₄₀-alkylaryl or —(R²—O)ₙ—R³;
R², R⁴: independently at each occurrence C₂-C₁₀-alkylene, C₆-C₁₀-arylene, or C₇-C₁₀-aralkylene;
R³: H, C₁-C₄₀-alkyl, C₃-C₁₀-cycloalkyl, C₆-C₁₀-aryl, C₇-C₄₀-aralkyl or C₇-C₄₀-alkylaryl;
n: 1 to 200;
m: 1 to 6;
p: 0 to 200;
*: linkage of the end group to the rest of the compound;
with the proviso that, for p=0, the end group, rather than via O, may also be linked to the rest of the compound via NR⁵, with R⁵ selected from H and C₁-C₃₀-alkyl.

In one embodiment of the invention, the compound is a polycondensate, i.e., a polymer whose monomer units are linked to one another with elimination of water. The polycondensate may have a linear, branched, hyperbranched or dendritic structure. Preferably the polycondensate has a linear structure or a structure with a low degree of branching.

As well as all polycondensates which within their molecules have only one kind of bonding between the monomer units, such as urethane bonding, urea bonding, ester bonding, amide bonding or carbonate bonding, for example, the polycondensates of the invention also include those polycondensates which within their molecules at the same time have at least two different forms of bonding between their monomer units, such as, for example, urethane bonding and ester bonding, or urethane bonding and urea bonding, or urea bonding and ester bonding.

Preferred compounds are selected from polyurethanes, polyureas, polyesters, polyamides, and polycarbonates, more preferably from polyurethanes, polycarbonates, polyesters, polyesterpolyurethanes, polyesterpolycarbonates, polyetherpolyurethanes, and polyetherpolycarbonates. The foregoing terms are known and have the meaning familiar to the skilled worker.

In accordance with the invention, however, the above-stated preferred compounds may each further comprise other kinds of bonding between the individual units as well. Thus, for example, polyurethanes, in addition to the urethane bonds resulting from the reaction between R—NCO and R′—OH, may also comprise urea bonds, resulting from the reaction between R—NCO and R′—NH.

Preferred compounds of the present invention are polyurethanes. Preferred polyurethanes are selected from polyetherurethanes and polyesterurethanes.

### Polyols

Polyols contain at least 2 OH groups. In one preferred embodiment of the invention, the polyurethanes of the invention comprise in copolymerized form at least one polyol, the polyol having a number-average molecular weight Mₙ in the range from 400 to 12 000 g/mol, more preferably from 400 to 10 000 g/mol.

Preferred polyols are polyester polyols and polyether polyols.

Polyesterpolyurethanes comprise as hydrophilic sections polyester polyols (also termed “polyesterols”) in cocondensed form. Polyester polyols comprise per molecule at least two OH groups and at least two ester groups; the number-average molar weight Mₙ of these compounds is preferably at least 400 g/mol. Polyester polyols preferred in accordance with the invention have a number-average molecular weight Mₙ in the range from 400 to 5000 g/mol, more preferably from 400 to 2000 g/mol.

Polyester polyols are prepared in general through the reaction of dicarboxylic acids with polyols at high temperature. Information on the industrial preparation of polyester polyols may be found, for example, in Kunststoffhandbuch Polyurethane, edited by G. Oertel, 3rd edn. 1993, Carl Hanser, section 3.1.2, especially section 3.1.2.3.

Polyetherpolyurethanes comprise as hydrophilic sections polyether polyols in cocondensed form. Polyether polyols (also “polyetherols”) comprise per molecule at least two OH groups and at least two ether groups, the Mₙ of these polyetherols being preferably at least 1000 g/mol.

The hydrophilicity of polyether polyols is generally such that they are water-soluble at room temperature (20° C.). The preparation of polyether alcohols is described in M. Ionescu, “Chemistry and technology of polyols for polyurethanes”, Rapra Technology, 2005.

Alkylene oxide starting products used frequently for the preparation of polyetherols are propylene oxide (PO) and/or ethylene oxide (EO).

Starter compounds contemplated for the preparation of polyetherols include, for example, the following:
a) monoethylene glycol, diethylene glycol, triethylene glycol, PEG, monopropylene glycol, dipropylene glycol, tripropylene glycol, polypropylene glycol, polytetrahydrofuran, glycerol, trimethylolpropane, trimethylolethane, neopentyl glycol, sugars and sugar derivatives such as sucrose or sorbitol, bisphenol A, bisphenol F, pentaerythritol, degraded starch, water, and mixtures thereof;
b) amines such as ethylenediamine, triethanolamine or tolylenediamine.

The alkylene oxide used is preferably propylene oxide, ethylene oxide, butylene oxide, isobutylene oxide, styrene oxide or mixtures of at least two of the stated alkylene oxides. As alkylene oxide it is preferred to use propylene oxide, ethylene oxide or mixtures of propylene oxide and ethylene oxide. Ethylene oxide is the alkylene oxide used with particular preference.

The operation may be carried out as a random copolymerization or as a block copolymerization, using different alkylene oxides.

Particularly suitable polyetherols are, for example, the products of polymerization of ethylene oxide (EO), the copolymerization or graft polymerization products thereof, and also the polyethers obtained by condensation of polyhydric alcohols or a mixture thereof, and the polyethers obtained by ethoxylation of polyfunctional alcohols, amides, polyamides, and amino alcohols. Examples thereof are, for instance, polyethylene glycols, adducts of ethylene oxide with trimethylolpropane, or EO-propylene oxide (PO) block copolymers.

Polyols suitable for preparing the polyurethanes of the invention are preferably those polyether polyols which comprise at least predominantly polyethylene glycol. Examples of suitable polyethylene glycols are those having an average EO unit content in the range from 30 to 450 per polymer molecule.

Other suitable polyether polyols are those having more than 2 hydroxyl groups. They are obtainable, for example, by addition of EO and/or PO with polyhydric alcohols such as glycerol, trimethylolpropane, trimethylolethane, sugars and sugar derivatives such as sucrose or sorbitol, pentaerythritol, or with amines. As a result of the free OH groups, such polyether polyols provide branching points within the compounds of the invention.

Preference is given to polyols of the general formula HO—(CH₂—CH₂—O)ₙ—H, where n can adopt the values in the range from 30 to 450. The polyetherols preferably possess Mₙ values in the range from 1500 to 12 000 g/mol, more preferably up to 10 000 g/mol.

In one preferred embodiment of the invention, then, the polyurethanes obtainable by the process of the invention comprise in copolymerized form at least one polyether polyol, said at least one polyether polyol having a number-average molecular weight Mₙ in the range from 1500 to 12 000 g/mol, more preferably from 4000 to 10 000 g/mol. With very particular preference the copolymerized polyetherols have a number-average molecular weight Mₙ in the range from 6000 g/mol to 9000 g/mol.

In one embodiment of the inventions the polyetherols to be copolymerized into the polyurethanes have a number-average molecular weight Mₙ in the range from 5800 to 6200 g/mol.

In another embodiment of the invention the polyetherols to be copolymerized into the polyurethanes have a number-average molecular weight Mₙ in the range from 8800 to 9200 g/mol.

The polyurethanes of the invention may also comprise, in copolymerized form, low molecular weight compounds having at least 2 hydroxyl groups or at least 2 amine groups. Low molecular weight compounds of this kind having 2 hydroxyl or amine groups are generally termed chain extenders, may be functionalized with ionic groups, and are known to the skilled worker. Low molecular weight compounds having more than 2 hydroxyl or amine groups serve as branching points within the compounds of the invention.

The polyurethanes of the invention may also comprise, in copolymerized form, relatively high molecular weight compounds having one hydroxyl group or one amine group. Examples of such compounds include polyetherols with all bar one of their OH groups etherified. Examples of such are the polyalkylene glycol alkyl ethers of the formula HO—(R²—O)ₙ-alkyl.

### Polyisocyanates

Suitable polyisocyanates comprise preferably on average 2 to a maximum of 4 NCO groups, with diisocyanates being particularly preferred.

Examples that may be given of suitable isocyanates include 1,5-naphthylene diisocyanate, 4,4′-diphenylmethane diisocyanate (MDI), hydrogenated MDI (H₁₂MDI), xylylene diisocyanate (XDI), tetramethylxylene diisocyanate (TMXDI), 4,4′-diphenyldimethylmethane diisocyanate, di- and tetraalkyldiphenylmethane diisocyanate, 4,4-dibenzyl diisocyanate, 1,3-phenylene diisocyanate, 1,4-phenylene diisocyanate, the isomers of tolylene diisocyanate (TDI), if desired as a mixture, 1-methyl-2,4-diisocyanatocyclohexane, 1,6-diisocyanato-2,2,4-trimethylhexane, 1,6-diisocyanato-2,4,4-trimethylhexane, 1-isocyanatomethyl-S-isocyanato-1-trimethylcyclohexane, 4,4′-diisocyanatophenylperfluoroethane, tetramethoxybutane 1,4-diisocyanate, butane 1,4-diisocyanate, hexane 1,6-diisocyanate (HDI), dicyclohexylmethane diisocyanate, cyclohexane 1,4-diisocyanate, ethylene diisocyanate, and bisisocyanatoethyl phthalate.

In one of the preferred embodiments, the polyurethanes of the invention comprise cycloaliphatic or aliphatic diisocyanate radicals, more preferably aliphatic diisocyanate radicals.

Examples of cocondensed aliphatic diisocyanates include the following: 1,4-butylene diisocyanate, 1,12-dodecamethylene diisocyanate, 1,10-decamethylene diisocyanate, 2-butyl-2-ethylpentamethylene diisocyanate, 2,4,4- or 2,2,4-trimethylhexamethylene diisocyanate, and, in particular, hexamethylene diisocyanate (hexane 1,6-diisocyanate, HDI).

Examples of cocondensed cycloaliphatic diisocyanates include the following: isophorone diisocyanate (IPDI), 2-isocyanatopropylcyclohexyl isocyanate, 4-methylcyclohexane 1,3-diisocyanate (H-TDI), and 1,3-bis(isocyanatomethyl)cyclohexane. Additionally, H₁₂-MDI or diisocyanates known as “saturated MDI”, such as, for example, 4,4′-methylenebis(cyclohexyl isocyanate) (alternatively also called dicyclohexylmethane 4,4′-diisocyanate) or 2,4′-methylenebis(cyclohexyl)diisocyanate, may be present as radicals in the polyurethanes of the invention.

For preparing the compounds of the invention it is of course also possible to use mixtures of the aforementioned diisocyanates, in order to prepare mixtures of different polyurethanes. One preferred embodiment uses isocyanates having a functionality of 2 (2 NCO groups per molecule).

Another embodiment uses isocyanates having a functionality of greater than 2 (more than 2 NCO groups per molecule). Polyurethanes having a structure branched on the isocyanate segment are then obtained.

In the preparation of the compounds of the invention, the components that are reactive toward the hydroxyl groups and amine groups are used in an amount such that the groups reactive toward the hydroxyl groups and amine groups are present in excess. The result of such an excess is that the end groups of the formula (I) can be linked to the rest of the compound through the reaction of a polyether of the formula (I) with the —OH-reactive and/or —NH-reactive groups.

In one preferred embodiment of the invention, the compounds of the invention are prepared using an amount of isocyanate groups which is greater than the amount of OH groups in the polyols and, optionally, chain extenders. Where the compounds of the invention are prepared using, as well as the end groups, for example, exclusively diols and diisocyanates, the diisocyanates are used in a molar excess.

In one embodiment of the invention, the ratio of NCO groups to —NCO-reactive groups when preparing the compounds of the invention is in the range from 1.2:1 to 2:1, preferably from 1.4:1 to 1.8:1, with the —NCO-reactive groups in the polyethers of the formula (I) being disregarded for the purpose of the calculation.

Preferred polyetherpolyurethanes comprise polyether polyols and polyisocyanates in cocondensed form.

The invention accordingly provides above-described polymers of the invention wherein the polymer is a polyetherpolyurethane and comprises each in copolymerized form:

a. at least one polyether polyol and

b. at least one polyisocyanate.

It is preferred for the molar ratio of NCO groups of the polyisocyanate to OH groups of the polyether polyol, prior to the polymerization, to be in the range from 1.2:1 to 2:1, preferably in the range from 1.4:1 to 1.8:1.

In one preferred embodiment of the invention, polyetherpolyurethanes of the invention comprise in copolymerized form as polyol at least one polyetherdiol and as polyisocyanate at least one diisocyanate.

In another embodiment of the invention, polyetherurethanes of the invention comprise in cocondensed form as polyol at least one polyetherdiol and at least one polyisocyanate having at least three isocyanate groups (—NCO). Polyetherpolyurethanes of the invention of this kind are branched at least on the hydrophobic sections, based on the at least trifunctional polyisocyanate.

In a further embodiment of the invention, polyetherpolyurethanes of the invention comprise as polyol at least one polyether polyol having at least three hydroxyl groups and as polyisocyanate at least one diisocyanate. Polyetherpolyurethanes of the invention of this kind are branched at least on the hydrophilic sections, based on the at least trifunctional polyether polyol.

In a further embodiment of the invention, polyetherpolyurethanes of the invention comprise in cocondensed form at least one polyether polyol having at least three hydroxyl groups and at least one polyisocyanate having at least three isocyanate groups.

In one embodiment of the invention, polyetherpolyurethanes of the invention have a hyperbranched or dendritic structure. The preparation of hyperbranched polyurethanes is described in WO 97/02304 or DE 199 04 444, for example.

One particularly preferred embodiment of the invention relates to polyurethanes of the invention which are linear or have a low degree of branching, where the polyisocyanate component and the polyol component used in preparing the polyurethanes of the invention have the following composition:

The polyisocyanate component comprises at least 80%, preferably at least 90%, more preferably at least 95%, and more particularly at least 99% by weight of diisocyanates.

The polyol component comprises at least 80%, preferably at least 90%, more preferably at least 95%, and more particularly at least 99% by weight of diols, the diols being selected preferably from polyetherdiols.

### Polyether Dendron

The compounds of the invention further comprise at least one end group of the general formula (I)

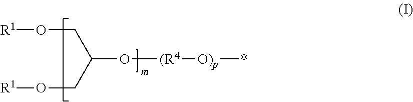

where the definitions are as follows:
R¹: independently at each occurrence C₄-C₄₀-alkyl, C₃-C₁₀-cycloalkyl, C₆-C₃₀-aryl, C₇-C₄₀-aralkyl, C₇-C₄₀-alkylaryl or —(R²—O)ₙ—R³;
R², R⁴: independently at each occurrence C₂-C₁₀-alkylene, C₆-C₁₀-arylene, or C₇-C₁₀-aralkylene;
R³: H, C₁-C₄₀-alkyl, C₃-C₁₀-cycloalkyl, C₆-C₁₀-aryl, C₇-C₄₀-aralkyl or C₇-C₄₀-alkylaryl;
n: 1 to 200;
m: 1 to 6;
p: 0 to 200;
*: linkage of the end group to the rest of the compound;
with the proviso that, for p=0, the end group, rather than via O, may also be linked to the rest of the compound via NR⁵, with R⁵ selected from H and C₁-C₃₀-alkyl.

R¹ is preferably C₁₀-C₃₂-alkyl, C₁₀-C₃₂-alkenyl, or —(R²—O)ₙ—R³. More preferably R¹ is C₁₀-C₃₂-alkenyl or —(R²—O)ₙ—R³. Very preferably R₁ is —(R²—O)ₙ—R³. In another preferred embodiment, R¹ is —(R²—O)ₙ—R³, where R² is C₂-C₅-alkylene and R³ is C₁₂-C₃₀-alkyl or C₇-C₄₀-aralkyl.

R² and R⁴, independently of one another, are preferably C₂-C₁₀-alkylene, substituted or preferably unsubstituted, examples being —CH₂—CH₂—, —CH(CH₃)—CH₂—, —CH₂—CH(CH₃)—, —CH(C₂H₅)—CH₂—, —CH₂—CH(C₂H₅)—, —(CH₂)₃—, —(CH₂)₄—, —(CH₂)₅—, —(CH₂)₆—, —(CH₂)₈—, —(CH₂)₁₀, preferably —CH₂—CH₂—, —CH(CH₃)—CH₂— or —CH₂—CH(CH₃)—, more preferably —CH₂—CH₂—. The alkylene radicals R² may be present individually or in mixtures of different alkylene radicals. For example, ethylene and propylene radicals may be mixed. The various alkylene radicals may be present in random order or in block form.

R³ is preferably H, C₁-C₂₈-alkyl, C₇-C₄₀-aralkyl, or C₇-C₄₀-alkylaryl. More preferably R³ is C₆-C₂₈-alkyl, selected for example from hexyl, heptyl, octyl, nonyl, decyl, undecyl, dodecyl, tridecyl, tetradecyl, pentadecyl, hexadecyl, heptadecyl, octadecyl, nonadecyl, icosyl, henicosyl, docosyl, tricosyl, tetracosyl, pentacosyl and/or hexacosyl, and very preferably C₁₂-C₂₆-alkyl, as for example dodecyl, tridecyl, tetradecyl, pentadecyl, hexadecyl, heptadecyl, octadecyl, nonadecyl, icosyl, henicosyl, docosyl, tricosyl, tetracosyl, pentacosyl and/or hexacosyl.

Branched isomers of these alkyl radicals, such as 2-ethylhexyl, isododecyl, isotetradecyl, isohexadecyl, isooctadecyl, and isoeicosyl, for example, may also be used, as may radicals of cycloalkanes or alkenes.

In one embodiment, R³ is C₈-C₂₀-alkyl and R² is ethylene.

In one embodiment, R² is ethylene and R³ is a C₁₋₃-alkyl, more particularly iso-C₁₃-alkyl.

n is preferably 2 to 100, more preferably 4 to 50, and very preferably 8 to 40.

m is preferably 2 to 5 and more preferably 3 to 4.

p is preferably 0 to 50, more preferably 0 to 20.

End groups of the general formula (I) are attached to the rest of the compound preferably by reaction of the corresponding polyether monoalcohol of the general formula (Ia)

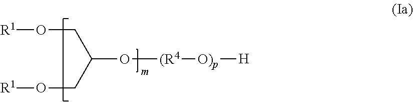

where R¹, R², R⁴, m, and p conform to the definitions of the general formula (I) with groups that are reactive toward OH groups.

Polyether dendrons of the formula (Ia) and their preparation are general knowledge, from EP 09165576.1, for example. For their preparation, at least one compound of the general formula R¹—OH is reacted with glycerol or, preferably, with a reactive glycerol derivative, more particularly with epichlorohydrin. For this reaction, it is preferred to select a molar ratio of the compound of the general formula R¹—OH to reactive derivative of glycerol, more particularly epichlorohydrin, of 2:1.

The preparation can be carried out, for example, at temperatures in the range from 20 to 200° C., preferably at 80 to 130° C.

The preparation can be carried out in the presence of a catalyst. Examples of suitable catalysts include inorganic and organic bases. Where epichlorohydrin is used as reactive glycerol derivative, base serves not only as catalyst but also for neutralizing the resultant HCl. Examples of suitable inorganic bases include alkali metal carbonates and, in particular, alkali metal hydroxides such as NaOH and KOH. Examples of suitable organic bases include tertiary amines, more particularly triethylamine and diazabicyclo[2.2.2]octane (DABCO), and also pyridine and para-N,N-dimethylaminopyridine.

In one embodiment, the preparation can be carried out in a solvent. Examples of suitable solvents include ethers, more particularly 1,4-dioxane, diisopropyl ether, tetrahydrofuran (“THF”), and di-n-butyl ether. Other suitable solvents are n-butyl acetate (“butyl acetate”), DMSO, N,N-dimethylformamide (“DMF”), and N-methylpyrrolidone, and aromatic solvents such as toluene, for example.

In embodiments where water is eliminated in the course of the preparation, water-removing agent can be used, an example being molecular sieve, sodium sulfate or magnesium sulfate, or the water formed may be removed by azeotropic distillation. In one embodiment of the present invention, the reaction is carried out over a time of 15 minutes to 48 hours, preferably 1 to 24 hours, more preferably 3 to 15 hours.

In one embodiment of the present invention, the reaction is carried out in stages, specifically in the number of stages corresponding to the desired m. In this kind of reaction, reactive derivative of glycerol, more particularly epichlorohydrin, is added in the relevant number of stages. For the staged reaction, an example of a possible procedure is one in which first a defined amount of compound of the general formula R¹—OH is reacted with half the number of moles of glycerol or, preferably, with a reactive derivative of glycerol, more particularly with epichlorohydrin. Subsequently, an amount of glycerol, or of reactive derivative of glycerol, that corresponds to a quarter of the number of moles of compound of the general formula R¹—OH is added, and reaction is carried out. If it is desired to carry out a further stage, then an amount of glycerol or of reactive derivative of glycerol corresponding to an eighth of the number of moles of compound of the general formula R¹—OH is subsequently added, and the reaction is carried out. At each further stage, the number of moles of compound of the general formula R¹—OH added is reduced correspondingly.

An advantageous feature of the synthesis route described is that after the individual stages there is no need for costly and inconvenient purification or separation of the reaction mixture.

A focal hydroxyl group is the single terminal OH group in the dendritic polyether of the formula (Ia) when p=0.

The formula (I) end groups with p=0 may have the following appearance, for example:

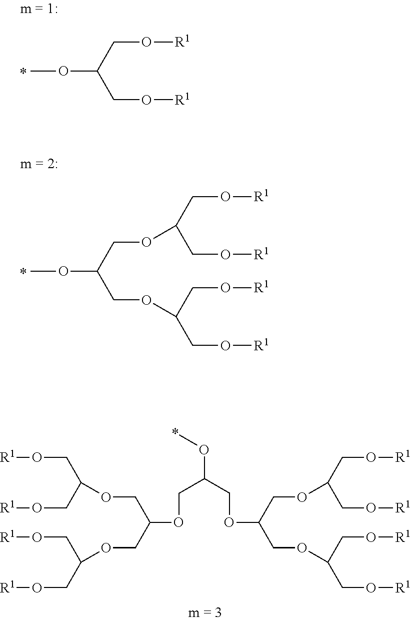

In one embodiment of the present invention, the end groups of the formula (I) are contaminated with products of an incomplete reaction. Owing to an incomplete reaction, the following structural units, for example, may then be formed:

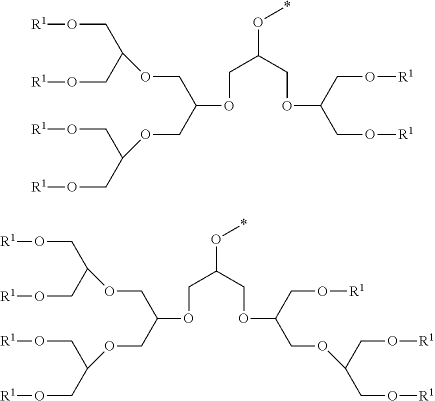

It is characteristic of this embodiment, then, that there are mixtures of dendrons with different indices m.

The end groups of the general formula (I) and the above-stated part-reacted end groups, in one embodiment of the invention, are attached to the rest of the compound either by an oxygen atom or via a nitrogen atom.

In a preferred embodiment of the invention, the end groups of the formula (I) and the part-reacted end groups are linked to the rest of the compound by an oxygen atom.

In one embodiment of the present invention, at least one end group of the general formula (I) is preferably attached to the rest of the compound via a structural unit selected from esters, amides, imides, urethanes, ureas, amines, and ethers.

In an embodiment of the present invention, at least one end group of the general formula (I) is more preferably attached to the rest of the compound via a structural unit selected from urethanes and ureas.

Compounds of the invention may comprise per molecule one or more end groups of the general formula (I).

End groups of the formula (I) and part-reacted end groups each with p>0 may be obtained, for example, by alkoxylation of the focal OH group of the corresponding polyether dendron of the formula (Ia) with p=0 and/or of the part-reacted polyether dendron with p=0.

Embodiments of the present invention further provide a process for preparing the compound of the invention, comprising at least the following steps:
A) providing the polyols and, if desired, reducing the water content of the polyols;
B) adding polyisocyanates and, if desired, catalyst, the amount of NCO groups in the resultant reaction mixture being greater than the amount of NCO-reactive groups;
C) reacting the reaction mixture resulting in step B);
D) reacting the reaction product from step B) with a polyether of the general formula (Ia)

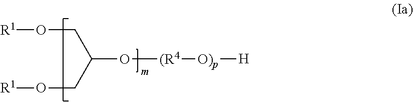

where the definitions are as follows:
R¹: independently at each occurrence C₄-C₄₀-alkyl, C₃-C₁₀-cycloalkyl, C₆-C₃₀-aryl, C₇-C₄₀-aralkyl, C₇-C₄₀-alkylaryl or —(R²—O)ₙ—R³;
R², R⁴: independently at each occurrence C₂-C₁₀-alkylene, C₆-C₁₀-arylene, or C₇-C₁₀-aralkylene;
R³: H, C₁-C₄₀-alkyl, C₃-C₁₀-cycloalkyl, C₆-C₃₀-aryl, C₇-C₄₀-aralkyl or C₇-C₄₀-alkylaryl;
n: 1 to 200;
m: 1 to 6;
p: 0 to 200.

In addition to the substances added in steps A to D it is also possible to add solvents.

In one embodiment of the invention, the reaction mixture obtained is admixed with water after the end of the reaction, thereby producing preferably an aqueous dispersion. “After the end of the reaction” denotes the point in time at which the isocyanate group content, based on the reaction mixture following addition of all reactive substances, is less than 5%, preferably less than 1%, and more preferably less than 0.1% by weight.

In one embodiment of the invention, the process of the invention uses as at least one polyol a polyetherol, preferably a polyetherdiol.

In one embodiment of the invention, the process of the invention uses as at least one polyisocyanate a diisocyanate.

In one embodiment of the invention, the reactants used include compounds having more than two hydroxyl groups per molecule and/or compounds having more than two isocyanate groups per molecule.

The use of such reactants in the preparation of the compounds of the invention results in further branches in these compounds. The compounds obtained, therefore, comprise further branches in the polymer backbone in addition to the dendron end groups.

The invention, therefore, also provides compounds of the invention which further to the branched groups of the general formula (I) comprise additional branches.

### Solvents

The process of certain embodiments of the invention can be carried out in the present of solvent. By solvents in the present case are meant substances or mixtures of substances that are liquid under standard conditions and in which, under standard conditions, at least one of the reactive components is soluble to an extent of at least 10% by weight, based on the resultant solution, its solubility being clear to the human eye and being without phase separation. By reactive components are meant all substances which, in the course of the preparation of the compounds of the invention, are incorporated chemically into the compounds of the invention. Examples of reactive components in the process of the invention are polyols, polyisocyanates, compounds having a hydroxyl group, and compounds having an isocyanate group.

In one embodiment of the invention, the fraction of the solvents, based on the total amount of all substances present during the process of the invention, is at least 30%, preferably at least 50%, more particularly at least 70% by weight.

In another embodiment of the invention, the fraction of the solvents, based on the total amount of all substances present during the process of the invention, is not more than 10%, preferably not more than 5%, more preferably not more than 1%, and more particularly not more than 0.1% by weight. One embodiment of the invention, therefore, is a substantially solvent-free process, also termed a melt process.

For preparing the compounds of the invention it is preferred to use polyol which as far as possible is anhydrous. The removal of the water from the polyol may take place in a step A by azeotropic distillation, drying under reduced pressure, or other methods known to the skilled worker. For example, by azeotropic distillation, water can be removed until the water content prior to the addition of the substances that are reactive toward hydroxyl groups, preferably polyisocyanates, is approximately 300 ppm.

Preparation for the reaction itself may involve, for example, either placing the polyol under reduced pressure and thus accomplishing sufficient removal of the water (preferably down to a water content of approximately 300 ppm or less), followed by admixing of a solvent, or else mixing the polyol with a solvent such as xylene, toluene or acetone and removing the water by azeotropic distillation down, for example, to a water content of approximately 300 ppm, in which case, however, the solvent is not completely removed—instead, the solution of polyol in the remaining solvent is used for the reaction in solution.

The compounds of certain embodiments of the invention can be prepared by a one-pot reaction or else in a multistage, preferably two-stage, process.

A one-pot reaction for the present purposes is a preparation process which comprises mixing substantially all of the required reactive substances and solvents before the beginning of the reaction in the reaction chamber (for example, round-bottom flask, three-neck flask, conical flask, reaction vessel, vessel cascade, flow tube) and then causing them to react (usually with stirred circulation/mixing and heating or cooling). If desired, individual components may also not be added until during the reaction.

Before the beginning of the reaction, preferably at least 90% by weight, more preferably at least 95% by weight, of all the reactive substances are present in the reaction chamber.

### Catalysts

Suitable catalysts include in principle all of the catalysts that are typically used in polyurethane chemistry. It is preferred to use the catalysts described in WO 2009/135857, pages 14-16. With particular preference the catalyst is selected from zinc 2-ethylhexanoate (also called zinc octanoate), zinc n-octanoate, zinc n-decanoate, zinc neodecanoate, zinc ricinoleate, and zinc stearate. More particularly, zinc neodecanoate is used.

### Cosmetic Preparations

The compounds of the invention are used preferably in cosmetic preparations. The invention therefore provides cosmetic preparations comprising the compounds of the invention.

One embodiment of the invention are water-comprising cosmetic preparations comprising the compounds of the invention.

The preparations of the invention may take the form of aqueous or aqueous-alcoholic solutions, O/W (preferably) and W/O emulsions, W/O/W (preferred) and O/W/O emulsions, hydrodispersion formulations, solids-stabilized formulations, stick formulations, PIT formulations, in the form of creams, foams, sprays (pump sprays or aerosols), gels, gel sprays, lotions, oils, oil gels or mousses, and may accordingly be formulated with customary further auxiliaries.

The preparations of the invention preferably take the form of a gel, foam, mousse, spray, ointment, cream, emulsion, suspension, lotion, milk or paste.

In one or more embodiments, the invention relates preferentially to cosmetic preparations selected from gels, gel creams, milks, hydroformulations, stick formulations, cosmetic oils and oil gels, mascara, self-tanning preparations, facecare compositions, bodycare compositions, and aftersun preparations. Cosmetic preparations are also understood to include preparations for oral care.

Further cosmetic preparations of the invention are skin cosmetic preparations, especially those for skincare. These preparations take the form more particularly of W/O or, preferably, O/W skin creams, day creams and night creams, eye creams, face creams, antiwrinkle creams, mimic creams, moisturizing creams, bleaching creams, vitamin creams, skin lotions, care lotions, and moisturizing lotions.

Further preferred preparations of the invention are face masks, cosmetic lotions, and preparations for use in decorative cosmetics, as for example for concealer sticks, stage makeup, mascara and eyeshadows, lipsticks, kohl pencils, eyeliners, make-ups, foundations, blushers, powders, and eyebrow pencils.

Further preparations of the invention are anti-acne compositions, repellents, shaving compositions, hair removal compositions, intimate care compositions, footcare compositions, and babycare products.

Further preferred preparations of the invention are washing, showering, and bathing products. Washing, showering, and bathing products for the purposes of this invention are soaps of liquid to gel-like consistency, transparent soaps, luxury soaps, deodorant soaps, cream soaps, baby soaps, skin protection soaps, abrasive soaps, and syndets, pasty soaps, soft soaps, and washing pastes, liquid washing, showering, and bathing products, such as washing lotions, shower lotions and shower gels, foam baths, oil baths, shower oils, and scrub products, and shaving foams, shaving lotions, and shaving creams.

Cosmetic preparations which comprise specific polyurethanes are described in WO 2009/135857, for example. The compounds of the present invention are generally also suitable for use in the preparations described in WO 2009/135857. The disclosure content of WO 2009/135857 is hereby incorporated by reference. In the context of the present invention, the polyurethanes used in the preparations of WO 2009/135857 are replaced by the compounds of the invention. The compounds of the invention, therefore, are used in the preparations of WO 2009/135857 preferably in place of the polyurethanes used therein.

Suitable ingredients for the preparations of the invention are described in WO 2009/135857, page 24 to page 35, hereby incorporated in full by reference.

Also in accordance with the invention are cosmetic UV light protection compositions comprising the compounds of the invention. Cosmetic light protection compositions are understood in the context of this invention to be cosmetic preparations which comprise at least one and preferably two or more UV filter substances.

UV light protection compositions corresponding to the UV light protection preparations of the invention are described in WO 2009/135857, page 35 to page 42, hereby incorporated in full by reference.

Embodiments of the invention also relate to cosmetic preparations, preferably in liquid or pastelike form, for use on the skin, on semimucosae, on mucosae, and more particularly on keratinic materials such as hair, eyelashes, and eyebrows, especially for the shaping, decorating, coloring, and beautifying thereof, and also for the care of the skin and of the epidermal derivatives. In principle the preparations of the invention, given appropriate formulation and coloration, may also be used as make-up, concealer, camouflage, eyeshadows, eyeliners, lipliners, blushers, lip blush, lip gloss, sun protection compositions, sunblock, temporary tattoos, colored effect sunscreen for surfers, and the like.

One preferred embodiment of the present invention, therefore, are decorative cosmetic preparations comprising the compounds of the invention.

Preparations corresponding to the decorative cosmetic preparations of the invention are described in WO 2009/135857, page 43 to page 46, hereby incorporated in full by reference.

According to one or more embodiments, the present invention provides aqueous preparations which in addition to the compounds further comprise at least one salt or surfactant or both.

A further embodiment of the invention are shampoos and cosmetic cleansing compositions comprising the compounds of the invention.

Preparations corresponding to the shampoos and cosmetic cleansing compositions of the invention are described analogously in WO 2009/135857, page 46 to page 55, hereby incorporated in full by reference.

A further embodiment of the invention are deodorants or antiperspirants comprising the compounds of the invention, more particularly deodorant lotions and deodorant or antiperspirant sticks, based on an oil-in-water dispersion or oil-in-water emulsion for the application of active compounds, more particularly water-soluble active compounds, to the skin.

Preparations corresponding to the deodorants and antiperspirants of the invention are described analogously in WO 2009/135857, page 55 to page 59, hereby incorporated in full by reference.

A further embodiment of the invention are hair colorants comprising the compounds of the invention.

Preparations corresponding to the hair colorants comprising the compounds of the invention are described analogously in WO 2009/135857, page 59 to page 65, hereby incorporated in full by reference.

Further embodiments of the invention are haircare compositions, more particularly hair conditioners, comprising the compounds of the invention.

Haircare compositions corresponding to the haircare compositions comprising the compounds of the invention are described analogously in WO 2009/135857, page 59 to page 67, hereby incorporated in full by reference.

Further embodiments of the invention are acidic preparations comprising the compounds of the invention.

Numerous cosmetic preparations comprise active compounds which develop their desired effect particularly at acidic pH values. Such preparations include, for example, preparations which comprise alpha-hydroxycarboxylic acids (AHA) and beta-hydroxycarboxylic acids (BHA), since in neutralized state these ingredients have little or no activity. Acidic preparations corresponding to the acidic preparations comprising the compounds of the invention are described analogously in WO 2009/135857, page 67 to page 69, hereby incorporated in full by reference.

Further embodiments of the invention are self-tanning products comprising the compounds of the invention.

Commercial self-tanning products generally constitute O/W emulsions. In these emulsions, the water phase is stabilized by emulsifiers customary in cosmetology.

Through application of the self-tanning products of the invention it is not only possible to achieve uniform skin coloration; it is also possible to provide uniform coloration to areas of skin that are differently colored by nature or as a result of morbid alteration.

Self-tanning substances used with advantage in accordance with the invention include glycerylaldehyde, hydroxymethylglyoxal, γ-dialdehyde, erythrulose, 5-hydroxy-1,4-naphthoquinone (juglone), and also 2-hydroxy-1,4-naphthoquinone as present in henna leaves. Especially preferred is 1,3-dihydroxyacetone (DHA), a trivalent sugar which occurs in the human body. 6-Aldo-D-fructose and ninhydrin as well can be used as self-tanning agents of the invention. Also considered self-tanning agents in the sense of the invention are substances which induce a skin coloration other than a brown shade.

In one preferred embodiment of the invention, such preparations comprise self-tanning substances in a concentration of 0.1% to 10% by weight and more preferably of 0.5% to 6% by weight, based in each case on the total weight of the preparation.

These preparations preferably comprise 1,3-dihydroxyacetone as self-tanning substance. With further preference these compositions comprise organic and/or inorganic light protection filters. The preparations may also comprise inorganic and/or organic and/or modified inorganic pigments.

Further ingredients preferably included in the preparations of the invention are specified, for example, in DE 103 21 147, in paragraphs [0024] to [0132], hereby incorporated in full by reference.

The invention further provides for the use of such preparations for coloring the skin of multicellular organisms, more particularly the skin of humans and animals, not least for harmonizing the color of differently pigmented areas of skin.

A further embodiment of the invention are preparations for oral and dental care and cleansing that comprise the compounds of the invention.

Oral and dental care and cleansing preparations corresponding to the preparations comprising the compounds of the invention are described analogously in WO 2009/135857, page 69 to page 70, hereby incorporated in full by reference.

A further embodiment of the invention are preparations for hair removal that comprise the compounds of the invention.

Hair removal preparations corresponding to the preparations comprising the compounds of the invention are described analogously in WO 2009/135857, page 70 to page 71, hereby incorporated in full by reference.

A further embodiment of the invention are preparations for permanent hair shaping that comprise the compounds of the invention.

Permanent hair shaping preparations corresponding to the preparations comprising the compounds of the invention are described analogously in WO 2009/135857, page 71 to page 73, hereby incorporated in full by reference.

Embodiments of the invention also provide the use of polymers for increasing compatibility with polar solvents, such as ethanol, propylene glycol or glycerol.

The invention likewise provides the use of polymers of the invention for increasing the solubility of sparingly water-soluble ingredients such as, for example, hydrophilic UV screens.

The invention further provides the use of polymers of the invention for increasing the water-binding capacity in the preparation and after application to the skin (moisturizer)

The invention likewise provides the use of polymers of the invention for improving the dispersibility of particles in the preparation.

### Experimental Section

Specific embodiments of the invention are described in more detail below by means of examples, without being confined to these examples.

Compound A.1 was prepared in 1,4-dioxane dried by standard methods. The molecular weight of A.1 was determined by gel permeation chromatography (GPC) in DMAC (N,N-dimethylacetamide) as solvent, using PMMA as standard.

The molecular weight of A.2/A.3 was determined by GPC in THF (tetrahydrofuran) as solvent, using PMMA as standard.

All of the reactions were carried out under an atmosphere of inert gas (dried nitrogen).

Amounts in % denote % by weight, unless expressly stated otherwise.

### Determination of Dynamic Viscosity

The dynamic viscosities of the compounds of the invention in aqueous dispersion were measured in the form of a 10 percent by weight dispersion at 23° C. The dynamic viscosity was determined at shear rates of 100 l/s and 350 l/s. These two values allow a conclusion to be drawn on whether the compounds of the invention exhibit structurally viscous (pseudoplastic) or newtonian thickener behavior in aqueous dispersion.

The dynamic viscosity was determined in accordance with DIN 53019 using:

Physica Rheolab MCI Portable rotational viscometer (Anton Paar);

cylinder measuring system, Z4 DIN cylinder (diameter 14 mm)

### Synthesis Example 1

### Preparation of the Polyether Dendron A.1

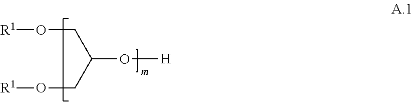 [0184] where
R¹=—(R²—O)ₙ—R³;
R²=—CH₂—CH₂—;
R³=approximately 1:1 parts by weight mixture of n-octadecyl (stearyl) and n-hexadecyl (cetyl);
n=25;
m=2.

A 1-liter flask with dropping funnel, magnetic stirrer, and reflux condenser was charged with a solution of 200 g (147 mmol) of an approximately 1:1 parts by weight mixture of polyethylene glycol monocetyl ether, CH₃—(CH₂)₁₅—O—(CH₂CH₂—O)₂₅H, and polyethylene glycol monostearyl ether, CH₃—(CH₂)₁₇—O—(CH₂CH₂—O)₂₅H (Lutensol® AT25, BASF SE), in 525 ml of dioxane. With stirring, 24 g of KOH pellets (428 mmol) were added. The mixture was heated to 100° C. and then 6.8 g (73.5 mmol) of epichlorohydrin, in solution in 25 ml of dioxane, were added dropwise over a period of 30 minutes. The mixture was then stirred for 2 hours, after which a further 3.4 g (36.8 mmol) of epichlorohydrin, in solution in 25 ml of dioxane, were added dropwise over a period of 30 minutes. This was followed by stirring for 2 hours more, after which, again, 1.7 g (18.4 mmol) of epichlorohydrin, in solution in 25 ml of dioxane, were added dropwise over a period of 15 minutes. The mixture was stirred at 105° C. for a period of a further 15 hours, and then was cooled to room temperature (25° C.). The precipitate formed was filtered off and the dioxane was distilled off at 30 mbar. This gave compound A.1 as a viscous light brown oil which was characterized by MS (MALDI TOF) and by GPC. Mₙ: 4400 g/mol, M_{w}: 6600 g/mol.

### Synthesis Example 2

### Preparation of a Polyetherpolyurethane A.2

60.00 g of polyethylene glycol Pluriol®E6000 (BASF SE, molecular weight 6000 g/mol) were dissolved under nitrogen in 334.00 g of xylene in a 2 l polymerization reactor. After the solution had been heated to approximately 140° C. (internal temperature), 200 g of xylene were distilled off. The water content of the reaction mixture was subsequently approximately 70 ppm. The polymer solution was then cooled to 50° C. (internal temperature) and admixed with 53 mg of acetic acid, in solution in 2.5 ml of xylene, in order to neutralize the quantity of potassium acetate, determined quantitatively beforehand, in the polyethylene glycol. By addition of 180 mg of zinc neodecanoate (TIB Kat 616), in solution in 2.5 ml of xylene, and 2.94 g of hexamethylene diisocyanate, in solution in 5 ml of xylene, the polymerization was initiated and the reaction mixture was allowed to react at an internal temperature of 50° C. until an isocyanate content of 0.25% by weight was reached. Then 66.0 g of the compound A.1, in solution in 66 ml of xylene, were added and the reaction mixture was heated further at 50° C. until the isocyanate content was 0%. The xylene solvent was largely removed (residual content <100 ppm) thereafter by vacuum distillation at elevated temperature (60° C.) and the residue was dissolved in 515.8 g of water. After the aqueous solution had been cooled to room temperature (25° C.), it was admixed, finally, with 6.45 g of the preservative Euxyl®K701 and with 70 mg of the stabilizer 4-hydroxy-TEMPO. This gave polymer A.2 (Mₙ=8700 g/mol; M_{w}=31 100 g/mol) in the form of an aqueous dispersion having a solids content of 18.7% by weight.

The viscosity of a 10% strength by weight aqueous solution of the polyetherpolyurethane A.2 was 20 500 mPa*s (shear rate 100 l/s) or 10 000 mPa*s (shear rate 350 l/s).

### Synthesis Example 3

### Preparation of a Polyetherpolyurethane A.3 (Comparative)

120.00 g of polyethylene glycol Pluriol®E6000 were dissolved under nitrogen in 467.00 g of xylene in a 2 l polymerization reactor. After the solution had been heated to approximately 140° C. (internal temperature), 200 g of xylene were distilled off. The water content of the reaction mixture was subsequently approximately 100 ppm. The polymer solution was then cooled to 50° C. (internal temperature) and admixed with 107 mg of acetic acid, in solution in 5 ml of xylene, in order to neutralize the quantity of potassium acetate, determined quantitatively beforehand, in the polyethylene glycol. By addition of 360 mg of zinc neodecanoate (TIB Kat 616), in solution in 5 ml of xylene, and 5.88 g of hexamethylene diisocyanate, in solution in 10 ml of xylene, the polymerization was initiated and the reaction mixture was allowed to react at an internal temperature of 50° C. until an isocyanate content of approximately 0.26% was reached. Then 40.8 g of an approximately 1:1 parts by weight mixture of polyethylene glycol monocetyl ether, CH₃—(CH₂)₁₅—O—(CH₂CH₂—O)₂₅H, and polyethylene glycol monostearyl ether, CH₃—(CH₂)₁₇—O—(CH₂CH₂—O)₂₅H (Lutensol® AT25, BASF SE), in solution in 40.8 ml of xylene, were added and the reaction mixture was heated further at 50° C. until the isocyanate content was 0%. The xylene solvent was largely removed (residual content <100 ppm) thereafter by vacuum distillation at elevated temperature (60° C.) and the residue was dissolved in 666.7 g of water. After the aqueous solution had been cooled to room temperature (25° C.), it was admixed, finally, with 8.34 g of the preservative Euxyl®K701 and with 80 mg of the stabilizer 4-hydroxy-TEMPO. This gave polymer A.3 (Mₙ=10 100 g/mol; M_{w}=35 000 g/mol) in the form of an aqueous dispersion having a solids content of 22.6% by weight.

The viscosity of a 10% strength by weight aqueous solution of the polyetherpolyurethane A.3 was 1750 mPa*s (shear rate 100 l/s) or 1550 mPa*s (shear rate 350 l/s).

TABLE 1
Viscosities of the thickeners as a function of shear rate

| | |
|---|---|
| Polymer | |
| concentration | Viscosity [mPa*s] |

| | in water [% | Shear rate | Shear rate |
|---|---|---|---|
| Polymer | by weight] | 100 1/s | 350 1/s |
| A.2 | 10 | 20 500 | 10 000 |
| A.3 | 10 | 1750 | 1550 |

The cosmetic formulations FA.2 and FA.3 were prepared by adding the oil phase A to the water phase B and subsequently admixing the resultant O/W emulsion with the preservative (phase C).

TABLE 2
Cosmetic formulations FA.2 and FA.3.

| Phase | Ingredients | FA.2 | FA.3* |
|---|---|---|---|
| Phase A | Cremophor ® A 6 | 2.0 g | 2.0 g |
| | Cremophor ® A 25 | 2.0 g | 2.0 g |
| | Lanette ® O | 2.5 g | 2.5 g |
| | Liquid paraffin | 5.0 g | 5.0 g |
| | Luvitol ® EHO | 5.0 g | 5.0 g |
| Phase B | Polyetherpoly- | A.2 | A.3 |
| | urethane | | |
| | | 0.5 g | 0.5 g |
| | 1,2-Propylene glycol | 5.0 g | 5.0 g |
| | Water | 77.5 g | 77.5 g |
| Phase C | Euxyl ® K701 | 0.5 g | 0.5 g |

TABLE 3
Viscosities of FA.2 and FA.3 at 2% by weight NaCl concentration

| | Viscosity [Pa*s] at |
|---|---|
| Formulation | NaCl concentration 2.0% |
| FA.2 | 14.0 |
| FA.3 | 8.9 |

Described below are typical preparations of the invention, but without the invention being confined to these examples.

The percentages are % by weight, unless expressly described otherwise.

Sunscreen cream 1

| | % | Ingredient | INCI |
|---|---|---|---|
| A | 58.7 | Water dem. | Aqua |
| | 0.1 | Edeta ® BD | Disodium EDTA |
| | 1.0 | Butylene Glycol | Butylene Glycol |
| | 2.0 | Uvinul ® MS 40 | Benzophenone-4 |
| | 1.0 | TEA | Triethanolamine |
| | 0.5 | Panthenol ® 75 W | Panthenol |
| | 2.4 | Polyetherpoly- | |
| | | urethane A.2 | |
| B | 5.0 | Neo Heliopan ® OS | Octyl Salicylate |
| | 3.0 | Eusolex ® 9020 | Avobenzone |
| | 5.0 | Neo Heliopan ® HMS | Homosalate |
| | 8.0 | Uvinul ® N 539 T | Octocrylene |
| | 1.0 | Cremophor ® GS 32 | Polyglyceryl-3 Distearate |
| | 1.0 | Cremophor ® A 6 | Ceteareth-6, Stearyl Alcohol |
| | 1.0 | Cremophor ® A 25 | Ceteareth-25 |
| | 2.0 | Lanette ® E | Sodium Cetearyl Sulfate |
| | 0.5 | Span ® 60 | Sorbitan Stearate |
| | 3.0 | Luvitol ® Lite | Hydrogenated Polyisobutene |
| | 2.0 | Lanette ® O | Cetearyl Alcohol |
| | 1.5 | Lanette ® 16 | Cetyl Alcohol |
| | 1.0 | Cetiol ® SB 45 | Butyrospermum Parkii |
| | | | (Shea Butter) |
| | 0.1 | Vitamin E Acetate | Tocopheryl Acetate |
| | 0.2 | Bisabolol rac. | Bisabolol |
| C | 0.5 | Glydant ® LTD | DMDM Hydantoin |

### Preparation

Heat phases A and B separately to approximately 80° C.

Stir phase B into phase A and briefly homogenize.

Cool to approximately 40° C. with stirring, add phase C, cool to room temperature with stirring, and again briefly homogenize.

Sunscreen cream 2

| | % | Ingredient | INCI |
|---|---|---|---|
| A | 2.0 | Cremophor ® A 6 | Ceteareth-6, Stearyl Alcohol |
| | 2.0 | Cremophor ® A 25 | Ceteareth-25 |
| | 5.0 | Luvitol ® EHO | Cetearyl Ethylhexanoate |
| | 5.0 | Liquid paraffin, | Mineral Oil |
| | | viscous | |
| | 2.5 | Lanette ® O | Cetearyl Alcohol |
| B | 5.0 | Z-Cote ® MAX | Zinc Oxide, Dimethoxydiphenylsilane/ |
| | | | Triethoxycaprylylsilane Crosspolymer |
| C | 2.4 | Polyetherpoly- | |
| | | urethane A.2 | |
| | 5.0 | 1,2-Propylene | Propylene Glycol |
| | | Glycol | |
| | 70.5 | Water, demin. | Water |
| D | 0.5 | Euxyl ® K 300 | Phenoxyethanol, Methylparaben, |
| | | | Ethylparaben, Butylparaben, |
| | | | Propylparaben |

### Preparation

Heat phase A to 80° C. Add phase B to phase A.

Homogenize phase A+B for 3 minutes.

Heat phase C to 80° C., stir into phase A+B, and homogenize.

Cool emulsion to 40° C. with stirring.

Add phase D, cool to room temperature with stirring, and homogenize.

Day cream with UV protection

| | | |
|---|---|---|
| % | Ingredient | INCI |

A

| | | |
|---|---|---|
| 3.00 | Tego Care ® 450 | Polyglyceryl-3 Methyl Glucose |
| | | Distearate |
| 3.00 | Lanette ® 18 | Stearyl Alcohol |
| 2.00 | Cutina ® GMS | Glyceryl Stearate |
| 4.00 | Estol ® 1540 | Ethylhexyl Cocoate |
| 5.00 | Luvitol ® EHO | Cetearyl Ethylhexanoate |
| 8.00 | Uvinul ® A Plus B | Ethylhexyl Methoxycinnamate, |
| | | Diethylamino Hydroxybenzoyl |
| | | Hexyl Benzoate |

B

| | | |
|---|---|---|
| 5.00 | D-Panthenol 50 P | Panthenol, Propylene Glycol |
| 0.10 | Edeta BD | Disodium EDTA |
| 1.0-5.0 | Polyetherpoly- | |
| | urethane A.2 | |
| ad 100 | Water dem. | Aqua dem. |

C

| | | |
|---|---|---|
| 0.20 | Bisabolol nat. | Bisabolol |
| q.s. | Perfume oil | |
| 0.50 | Aloe Vera Gel | Water, Aloe Barbadensis Leaf |
| | concentrate 10/1 | Juice |
| 0.50 | Euxyl ® K 300 | Phenoxyethanol, Methylparaben, |
| | | Butylparaben, Ethylparaben, |
| | | Propylparaben, Isobutylparaben |

### Preparation

Heat phases A and B separately to approximately 80° C.

Stir phase B into phase A and briefly homogenize.

Cool to approximately 40° C. with stirring, add phase C, cool to room temperature with stirring, and again briefly homogenize.

Make up

| | | |
|---|---|---|
| % | Ingredient | INCI |

A

| | | |
|---|---|---|
| 4.00 | Dracorin ® 100 SE | Glyceryl Stearate, PEG-100 Stearate |
| 1.00 | Uvinul ® A Plus | Diethylamino Hydroxybenzoyl Hexyl |
| | | Benzoate |
| 3.00 | Uvinul ® MC 80 | Ethylhexyl Methoxycinnamate |
| 0.50 | Emulmetik ® 100 | Lecithin |
| 0.50 | Rylo ® PG 11 | Polyglyceryl Dimer Soyate |

B

| | | |
|---|---|---|
| 0.35 | Sicovit ® Brown | Iron Oxides |
| | 75 E 172 | |
| 2.00 | Sicovit ® Red | Iron Oxides |
| | 30 E 172 | |
| 1.00 | Sicovit ® Yellow | Iron Oxides |
| | 10 E 172 | |
| 2.25 | Prisorine ® 3630 | Trimethylolpropane Triisostearate |

C

| | | |
|---|---|---|
| 5.50 | Dow Corning ® 345 | Cyclopentasiloxane, Cyclohexasiloxane |
| | Fluid | |
| 4.00 | Tegosoft ® OP | Ethylhexyl Palmitate |
| 1.50 | Jojoba oil | Simmondsia Chinensis (Jojoba) |
| | | Seed Oil |
| 2.00 | Miglyol ® 840 | Propylene Glycol Dicaprylate/Dicaprate |
| 1.50 | Almond oil, sweet | Sweet Almond |
| | | (Prunus Amygdalus Dulcis) |
| | | Oil |
| 0.50 | Vitamin E Acetate | Tocopheryl Acetate |
| 1.00 | Cetiol ® SB 45 | Butyrospermum Parkii (Shea Butter) |
| 5.00 | Uvinul ® TiO2 | Titanium Dioxide, |
| | | Trimethoxycaprylylsilane |
| 0.50 | Dehymuls ® PGPH | Polyglyceryl-2 Dipolyhydroxystearate |

D

| | | |
|---|---|---|
| 5.00 | 1,2-Propylene | Propylene Glycol |
| | glycol Care | |
| 0.50 | Lutrol ® F 68 | Poloxamer 188 |
| 0.10 | Edeta BD | Disodium EDTA |
| 1.0-5.0 | Polyetherpoly- | |
| | urethane A.2 | |
| ad 100 | Water dem. | Aqua dem. |

E

| | | |
|---|---|---|
| 1.00 | Euxyl ® K 300 | Phenonip |
| | | Phenoxyethanol, Methylparaben, |
| | | Ethylparaben, Butylparaben, |
| | | Propylparaben, Isobutylparaben |
| 0.20 | Bisabolol rac. | Bisabolol |
| q.s. | Perfume oil | |

### Preparation

Heat phases A, B, C and D separately from one another to 70° C.

Homogenize phase B on a triple-roll mill. Stir phase B into phase A.

Briefly homogenize the whole again.

Dissolve phase C and stir into phase A+B.

Dissolve phase D, stir into combined phases A+B+C, and homogenize.

Cool to approximately 40° C. with stirring, add phase E, and cool to room temperature. Briefly homogenize.

Tinted day cream

| | | |
|---|---|---|
| % | Ingredient | INCI |

A

| | | |
|---|---|---|
| ad 100 | Water dem. | Aqua dem. |
| 5.00 | Glycerol 87% | Glycerin |
| 4.00 | D-Panthenol 50 P | Panthenol, Propylene Glycol |
| 0.75 | Cloisonné ® Gold | Mica, Titanium Dioxide, Iron Oxides |
| 0.25 | Cloisonné ® Super | Mica, Iron Oxides |
| | Rouge | |
| 1.0-5.0 | Polyetherpoly- | |
| | urethane A.2 | |

B

| | | |
|---|---|---|
| 3.00 | Uvinul ® A Plus | Diethylamino Hydroxybenzoyl Hexyl |
| | | Benzoate |
| 7.00 | Luvitol ® Lite | Hydrogenated Polyisobutene |
| 1.50 | Lanette ® O | Cetearyl Alcohol |
| 1.50 | Cutina ® GMS | Glyceryl Stearate |
| 3.50 | Cetiol ® SB 45 | Butyrospermum Parkii (Shea Butter) |
| 3.50 | Olive oil | Olive (Olea Europaea) Oil |
| 1.00 | Eumulgin ® B 2 | Ceteareth-20 |
| 1.00 | Cremophor ® A6 | Ceteareth-6, Stearyl Alcohol |
| 1.00 | Cetiol ® OE | Dicaprylyl Ether |
| 0.05 | BHT | BHT |

C

| | | |
|---|---|---|
| 0.20 | Sodium Ascorbyl | Sodium Ascorbyl Phosphate |
| | Phosphate | |
| 5.00 | Water dem. | Aqua dem. |

D

| | | |
|---|---|---|
| 1.00 | Euxyl ® PE 9010 | Phenoxyethanol, Ethylhexylglycerin |
| 0.25 | Bisabolol rac. | Bisabolol |
| 1.00 | Vitamin E Acetate | Tocopheryl Acetate |
| q.s. | Perfume oil | |

### Preparation

Heat phase A to 80° C.

Heat phase B to approximately 80° C. and introduce with stirring into phase A. Homogenize.

Cool to approximately 40° C. with stirring, add phase C+D, and cool to room temperature with stirring.

Briefly homogenize.

Deodorant lotion

| | | |
|---|---|---|
| % | Ingredient | INCI |

A

| | | |
|---|---|---|
| 1.50 | Cremophor ® A 6 | Ceteareth-6, Stearyl Alcohol |
| 1.50 | Cremophor ® A 25 | Ceteareth-25 |
| 2.00 | Cremophor ® CO 40 | PEG-40 Hydrogenated Castor Oil |
| 2.00 | Cutina ® GMS | Glyceryl Stearate |
| 2.00 | Lanette ® O | Cetyl Alcohol |
| 2.00 | Softisan ® 100 | Hydrogenated Coco-Glycerides |
| 8.00 | Cetiol ® V | Decyl Oleate |
| 0.50 | Abil ® B 8843 | PEG-14 Dimethicone |
| 0.30 | Farnesol | Farnesol |

B

| | | |
|---|---|---|
| q.s. | Preservative | Preservative |
| 1.0-5.0 | Polyetherpoly- | |
| | urethane A.2 | |
| ad 100 | Water dem. | Aqua dem. |

C

| | | |
|---|---|---|
| q.s. | Perfume oil | Fragrance |

D

| | | |
|---|---|---|
| 5-20 | Locron ® L | Aluminum Chlorohydrate |

### Preparation

Heat phases A and B separately to approximately 80° C.

Stir phase B into phase A with homogenization, and with brief subsequent homogenization.

Cool to approximately 40° C., add phases C and D with homogenization, and leave to cool to room temperature with stirring.

Hair wax with pigments

| % | Phase | Ingredient | INCI |
|---|---|---|---|
| 5 | A | Cremophor ® CO | Hydrogenated Castor Oil |
| | | 40PEG-40 | |
| 15 | | Cremophor ® A 25 | Ceteareth-25 |
| 0.5-10 | | Polyetherpoly- | |
| | | urethane A.2 | |
| 15 | | Luvitol ® Lite | Hydrogenated Polyisobutene |
| 3 | | Marlipal ® MG | Laureth-7 |
| 2 | | Brij ® 98 | Oleth-20 |
| 1 | | Euxyl ® PE 9010 | Phenoxyethanol and |
| | | | Ethylhexylglycerin |
| 5 | B | Abil ® B 88183 | PEG/PPG-20/6 Dimethicone |
| ad 100 | | Water dem. | Water dem |
| 1 | | Gemtone ® Emerald | Mica and Titanium Dioxide and |
| | | | Chromium Oxide Greens and |
| | | | Ferric Ferrocyanide |

Preparation:

I: Weigh out phases A and B separately and heat to 80° C. with stirring
II: Combine phases A and B at 80° C. with stirring
III: Cool to room temperature with stirring

Hair gel with UV protection

| Phase % | | INCI | Ingredient |
|---|---|---|---|
| A | 44.55 | Aqua dem. | |
| | 0.45 | Acrylates/C10-30 Alkyl | |
| | | Acrylate Crosspolymer | |
| B | 0.36 | Aminomethyl Propanol | |
| C | 0.66 | Panthenol | D-Panthenol 75W ® |
| | 10.00 | PVP/VA Copolymer | Luviskol ® VA 64 W |
| | 2.50 | Polyquaternium-46 | Luviquat ® Hold |
| | 5.00 | Sorbitol | |
| | 0.10 | Disodium EDTA | |
| | 0.5 | Benzophenone-4 | Uvinul ® MS 40 |
| | q.s. | Perfume | |
| | q.s. | PEG-40 Hydrogenated | Cremophor ® CO 40 |
| | | Castor Oil | |
| | q.s. | Preservative | |
| | 5.00 | Alcohol | |
| | ad 55 | Aqua dem. | |
| | 0.5-4 | Polyetherpoly- | |
| | | urethane A.2 | |

### Preparation:

I: Weigh out phases A, B, and C separately and stir if necessary until homogeneous at room temperature
II: Combine phases B and A with stirring at room temperature, stir until homogeneous, then add phase C with stirring and stir until smooth

Hair mousse

| % | | Ingredient | INCI |
|---|---|---|---|
| qs | Phase A | Deionized Water | (Aqua dem.) |
| 11.00 | | Luviquat ® Hold | (Polyquaternium 46) |
| 1.50 | | Uvinul ® MS 40 | (Benzophenone-4) |
| | | | 20% sol., neutr. with |
| | | | triethanolamine |
| 0.1-3 | | Polyetherpoly- | |
| | | urethane A.2 | |
| 0.40 | | D,L Panthenol 50W | (Panthenol) |
| 0.20 | | Masil ® SF 19 CG | (PEG-8 Methicone) |
| 0.40 | | Glydant ® Plus | (DMDM Hydantoin (and) |
| | | liquid | Iodopropynyl |
| | | | Butylcarbamate) |
| 0.20 | Phase B | Cremophor ® CO 40 | (PEG-40 Hydrogenated |
| | | | Castor Oil) |
| 0.40 | | Vitamin E Acetate | (Tocopheryl Acetate) |
| 0.20 | | Bell ® 6101232 | (Fragrance) |
| 0.70 | | Rhodasurf ® L-4 | (Laureth-4) |
| 6.00 | Phase C | Propellant gas A46 | (Propane/Isobutane) |

### Preparation:

1. Weigh out the compounds of phase A with stirring until fully dissolved, in the order listed
2. Weigh out the compounds of phase B with stirring and heating at 40-45° C.
3. Combine phases A and B and transfer to suitable propellant gas containers for hair mousses
4. Seal and fill with propellant gas phase C.

Face cream with 3% Sodium Ascorbyl Phosphate

| | | |
|---|---|---|
| % | Ingredient | INCI |

A

| | | |
|---|---|---|
| 2.00 | Cremophor ® A 6 | Ceteareth-6, Stearyl Alcohol |
| 2.00 | Cremophor ® A 25 | Ceteareth-25 |
| 3.00 | Jojoba oil | Simmondsia Chinensis (Jojoba) |
| | | Seed Oil |
| 3.00 | Lanette ® O | Cetearyl Alcohol |
| 10.00 | Liquid Paraffin, | Mineral Oil |
| | viscous | |
| 5.00 | Vaseline | Petrolatum |
| 4.00 | Miglyol ® 812 | Caprylic/Capric Triglyceride |

B

| | | |
|---|---|---|
| 5.00 | 1,2-Propylene | Propylene Glycol |
| | glycol Care | |
| 0.10 | Edeta BD | Disodium EDTA |
| 1.0-5.0 | Polyetherpoly- | |
| | urethane A.2 | |
| 0.30 | Abiol ® | Imidazolidinyl Urea |
| ad 100 | Water dem. | Aqua dem. |

C

| | | |
|---|---|---|
| 0.08 | Sodium hydroxide | Sodium Hydroxide |

D

| | | |
|---|---|---|
| 0.50 | Vitamin E Acetate | Tocopheryl Acetate |
| 0.20 | Phenoxyethanol | Phenoxyethanol |
| 3.00 | Sodium Ascorbyl | Sodium Ascorbyl Phosphate |
| | Phosphate | |

### Preparation

Heat phases A and B separately to approximately 80° C.

Stir phase B into phase A and homogenize.

Stir phase C into phase A+B and homogenize.

Cool with stirring to approximately 40° C.

Stir in phase C and briefly homogenize.

Cool to room temperature with stirring.

Reduction in average droplet size distribution/O/W emulsion

| | | |
|---|---|---|
| % | Ingredient | INCI |

Phase A

| | | |
|---|---|---|
| 2.0 | Lanette ® O | Cetearyl Alcohol |
| 5.0 | Finsolv ® TN | C12-15 Alkyl Benzoate |
| 10.0 | Miglyol ® 812 | Caprylic/Capric Triglyceride |
| 5.0 | Cetiol ® B | Dibutyl Adipate |
| 2.0 | Amphisol ® K | Potassium Cetyl Phosphate |
| 0.5 | Elfacos ® ST-9 | PEG-45/Dodecyl Glycol Copolymer |

Phase B

| | | |
|---|---|---|
| 5.0 | 1,2-Propylene | Propylene Glycol |
| | glycol Care | |
| ad 100 | Water demin. | Water |
| 1.0-5.0 | Polyetherpoly- | |
| | urethane A.2 | |

Phase C

| | | |
|---|---|---|
| 0.5 | Euxyl ® K 300 | Phenoxyethanol, Methylparaben, |
| | | Ethylparaben, Butylparaben, |
| | | Propylparaben |

Heat phases A and B to approximately 80° C. Stir phase B into phase A, homogenize. Stir until cold, stir in phase C, briefly homogenize again.

AHA cream

| | | |
|---|---|---|
| % | Ingredient | INCI |

A

| | | |
|---|---|---|
| 2.00 | Cremophor ® A 6 | Ceteareth-6, Stearyl Alcohol |
| 2.00 | Cremophor ® A 25 | Ceteareth-25 |
| 8.00 | Liquid paraffin, | Mineral Oil |
| | viscous | |
| 7.00 | Luvitol ® EHO | Cetearyl Ethylhexanoate |
| 6.00 | Cutina ® GMS | Glyceryl Stearate |
| 1.00 | Lanette ® 16 | Cetyl Alcohol |
| 0.20 | Abil ® 350 | Dimethicone |
| 0.20 | Bisabolol nat. | Bisabolol |

B

| | | |
|---|---|---|
| 1.00 | D-Panthenol USP | Panthenol |
| 3.00 | 1,2-Propylene | Propylene Glycol |
| | glycol Care | |
| 1.0-5.0 | Polyetherpoly- | A.2 |
| | urethane | |
| 5.00 | Hydroxy acid* | |
| q.s. | Sodium hydroxide | Sodium Hydroxide |
| q.s. | Preservative | Preservative |
| ad 100 | Water dem. | Aqua dem. |

C

| | | |
|---|---|---|
| q.s. | Perfume oil | Fragrance |

*examples of hydroxy acids which can be used are as follows:
alpha-hydroxy acids: lactic acid, citric acid, malic acid, glycolic acid
dihydroxy acid: tartaric acid
beta-hydroxy acid: salicylic acid

### Preparation

Heat phases A and B separately to approximately 80° C. Optionally, adjust pH of phase B to 3 using NaOH.

Stir phase B into phase A with homogenization, briefly homogenize again.

Cool to approximately 40° C., add phase C, homogenize again.

Cream with vitamin A acid

| | | |
|---|---|---|
| % | Ingredient | INCI |

A

| | | |
|---|---|---|
| 1.50 | Cremophor ® A 25 | Ceteareth-25 |
| 1.50 | Cremophor ® A 6 | Ceteareth-6, Stearyl Alcohol |
| 3.00 | Tegin ® | Glyceryl Stearate SE |
| 2.00 | Lanette ® O | Cetearyl Alcohol |
| 10.00 | Luvitol ® EHO | Cetearyl Ethylhexanoate |
| 5.00 | Liquid paraffin, | Mineral Oil |
| | viscous | |
| 0.10 | D,L-Alpha-Tocopherol | Tocopherol |
| 0.10 | Vitamin A acid | Tretionin |

B

| | | |
|---|---|---|
| 1.0-5.0 | Polyetherpoly- | A.2 |
| | urethane | |
| 4.00 | 1,2-Propylene | Propylene Glycol |
| | glycol Care | |
| 0.10 | Edeta BD | Disodium EDTA |
| q.s. | Preservative | Preservative |
| ad 100 | Water dem. | Aqua dem. |

C

| | | |
|---|---|---|
| 0.40 | Triethanolamine Care | Triethanolamine |
| 3.00 | Vitamin E Acetate | Tocopheryl Acetate |
| 0.10 | Vitamin A acid | Tretionin |
| q.s. | Perfume oil | Fragrance |

### Preparation

Heat phase A and phase B separately to approximately 75° C. Stir phase B into phase A and homogenize. Stir until cold. Add phase C at approximately 30° C.

Note: The formulation is prepared without inert gas. Filling must take place into oxygen-impermeable packs, e.g., aluminum tubes.

Epilation cream 1

| | | |
|---|---|---|
| % | Ingredient | INCI |

A

| | | |
|---|---|---|
| 4.20 | Lanette ® 16 | Cetyl Alcohol |
| 1.26 | Brij ® 35 | Laureth-23 |

B

| | | |
|---|---|---|
| 15.00 | Luviquat ® Care | Polyquaternium-44 |
| 0.90 | D-Panthenol USP | Panthenol |
| 0.35 | Allantoin | Allantoin |
| q.s. | Preservative | Preservative |
| 22.40 | Calcium carbonate | Calcium Carbonate |
| 10.00 | Calcium hydroxide | Calcium Hydroxide |
| 5.40 | Calcium thioglycolate | Calcium Thioglycolate |
| ad 100 | Water dem. | Aqua dem. |
| 1.0-5.0 | Polyetherpoly- | A.2 |
| | urethane | |

C

| | | |
|---|---|---|
| q.s. | Perfume oil | Fragrance |

### Preparation

Heat phases A and B separately to approximately 80° C.

Stir phase B into phase A with homogenization, briefly homogenize again.

Cool to about 40° C., add phase C, homogenize again.

Epilation cream 2

| | | |
|---|---|---|
| % | Ingredient | INCI |

A

| | | |
|---|---|---|
| 1.00 | Cremophor ® A 6 | Ceteareth-6, Stearyl Alcohol |
| 1.00 | Cremophor ® A 25 | Ceteareth-25 |
| 4.00 | Lanette ® O | Cetearyl Alcohol |
| 6.00 | Liquid paraffin, | Mineral Oil |
| | viscous | |
| q.s. | Preservative | Preservative |

B

| | | |
|---|---|---|
| 8.00 | Calcium thioglycolate | Calcium Thioglycolate |
| 2.00 | 1,2-Propylene | Propylene Glycol |
| | glycol Care | |
| 1.0-5.0 | Polyetherpoly- | A.2 |
| | urethane | |
| 1.00 | Sodium hydroxide | Sodium Hydroxide |
| ad 100 | Water dem. | Aqua dem. |

C

| | | |
|---|---|---|
| q.s. | Perfume oil | Fragrance |

### Preparation

Heat phases A and B separately to approximately 80° C.

Stir phase B into phase A with homogenization, briefly homogenize again.

Cool to approximately 40° C., add phase C, homogenize again.

Conditioner Shampoo 1

| | | |
|---|---|---|
| 35.70 | g | Sodium Laureth Sulfate |
| 6.50 | g | Cocamidopropyl Betaine |
| 0.20 | g | Polyetherpolyurethane A.2 |
| 0.50 | g | Polyquaternium-7, PQ-10, PQ-39, PQ-44, PQ-67, |
| | | Guarhydroxypropyltrimonium Chloride and/or PQ-87 |
| 0.10 | g | Preservative |
| 0.10 | g | Perfume oil/essential oil |
| ad 100 | g | Aqua dem. |

Conditioner Shampoo 2

| | | |
|---|---|---|
| 35.70 | g | Sodium Laureth Sulfate |
| 6.50 | g | Cocamidopropyl Betaine |
| 0.50 | g | Polyetherpolyurethane A.2 |
| 0.20 | g | Guarhydroxypropyltrimonium Chloride |
| 0.10 | g | Preservative |
| 0.10 | g | Perfume oil/essential oil |
| ad 100 | g | Aqua dem. |

Hair Conditioner

| | | |
|---|---|---|
| 4.00 | g | Distearoyethyl Hydroxyethylmonium Methosulfate, |
| | | Cetearyl Alcohol |
| 1.00 | g | Hydrogenated Polyisobutene |
| 1.00 | g | Cyclopentasiloxane |
| 1.00 | g | Isopropyl Palmitate |
| 3.00 | g | Cetearyl Alcohol |
| 0.10 | g | Acrylate C10-30 Alkyl acrylate copolymer |
| 0.25 | g | Panthenol |
| 0.20 | g | Hydroxyethyl Cellulose |
| 2.00 | g | Propylene Glycol |
| 2.50 | g | Polyetherpolyurethane A.2 |
| 0.50 | g | Cationic polymer, such as Polyquaternium-7, PQ-10, |
| | | PQ-16, PQ-39, PQ-44, PQ-46, PQ-67, |
| | | Guarhydroxypropyltrimonium Chloride and/or PQ-87 |
| 0.10 | g | Preservative |
| 0.10 | g | Perfume oil/essential oil |
| ad 100 | g | Aqua dem. |

Shampoo 1
Phase A

| 15.00 | g | Cocamidopropyl Betaine |
|---|---|---|
| 10.00 | g | Disodium Cocoamphodiacetate |
| 5.00 | g | Polysorbate 20 |
| 5.00 | g | Decyl Glucoside |
| 0.50 | g | Polyquaternium-7, PQ-10, PQ-39, PQ-44, PQ-67, |
| | | Guarhydroxypropyltrimonium Chloride and/or PQ-87 |
| 0.20 | g | Polyetherpolyurethane A.2 |
| 0.10 | g | Perfume oil/essential oil |
| q.s. | | Preservative |
| 2.00 | g | Laureth-3 |

| | |
|---|---|
| ad 100 | Aqua dem. |
| q.s. | Citric Acid |

Phase B

| | | |
|---|---|---|
| 3.00 | g | PEG-150 Distearate |

### Preparation:

Weigh out and dissolve components of phase A; adjust pH to 6-7. Add phase B and heat to 50° C. Leave to cool to room temperature with stirring.

Shampoo 2

| 30.00 | g | Sodium Laureth Sulfate |
|---|---|---|
| 6.00 | g | Sodium Cocoamphoacetate |
| 0.50 | g | Polyquaternium-7, PQ-10, PQ-39, PQ-44, PQ-67, |
| | | Guarhydroxypropyltrimonium Chloride and/or PQ-87 |
| 0.50 | g | Polyetherpolyurethane A.2 |
| 3.00 | g | Sodium Laureth Sulfate, Glycol Distearate, Cocamide MEA, |
| | | Laureth-10 |
| 2.00 | g | Dimethicone |
| q.s. | | Perfume |
| q.s. | | Preservative |
| q.s. | | Citric Acid |
| 1.00 | g | Sodium Chloride |

| | |
|---|---|
| ad 100 | Aqua dem. |

Shower gel 1

| 20.00 | g | Ammonium Laureth Sulfate |
|---|---|---|
| 15.00 | g | Ammonium Lauryl Sulfate |
| 0.50 | g | Polyetherpolyurethane A.2 |
| 0.50 | g | Polyquaternium-10, PQ-22, PQ-44, PQ-67, |
| | | Guarhydroxypropyltrimonium Chloride and/or PQ-87 |
| 2.50 | g | Sodium Laureth Sulfate, Glycol Distearate, Cocamide MEA, |
| | | Laureth-10 |
| 0.10 | g | Perfume oil/essential oil |
| q.s. | | Preservative |
| 0.50 | g | Sodium Chloride |

| | |
|---|---|
| ad 100 | Aqua dem. |

Shower gel 2

| 40.00 | g | Sodium Laureth Sulfate |
|---|---|---|
| 5.00 | g | Decyl Glucoside |
| 5.00 | g | Polyetherpolyurethane A.2 |
| 1.00 | g | Panthenol |
| 0.50 | g | Polyquaternium-10, PQ-44, PQ-67, |
| | | Guarhydroxypropyltrimonium Chloride and/or PQ-87 |
| 0.10 | g | Perfume oil/essential oil |
| q.s. | | Preservative |
| q.s. | | Citric Acid |
| 2.00 | g | Sodium Chloride |

| | |
|---|---|
| ad 100 | Aqua dem. |

Shampoo 3

| 12.00 | g | Sodium Laureth Sulfate |
|---|---|---|
| 1.50 | g | Decyl Clucoside |
| 0.50 | g | Polyquaternium-10, PQ-44, PQ-67, |
| | | Guarhydroxypropyltrimonium Chloride and/or PQ-87 |
| 0.50 | g | Polyetherpolyurethane A.2 |
| 5.00 | g | Coco-Glucoside Glyceryl Oleate |
| 2.00 | g | Sodium Laureth Sulfate, Glycol Distearate, Cocomide MEA, |
| | | Laureth-10 |
| q.s. | | Preservative |
| q.s. | | Sunset Yellow C.I. 15 985 |
| 0.10 | g | Perfume oil/essential oil |
| 1.00 | g | Sodium Chloride |

| | |
|---|---|
| ad 100 | Aqua dem. |

The compounds of the invention can also be used in hair styling preparations, more particularly hair mousses (aerosol mousses with propellant gas, and pump mousses without propellant gas), hair sprays (pump sprays without propellant gas), and hair gels.

Propellants are the commonly used propellants. Preference is given to mixtures of propane/butane, pentane, dimethyl ether, 1,1-difluoroethane (HFC-152 a), carbon dioxide, nitrogen or compressed air.

Aerosol hair mousse

| | |
|---|---|
| 2.00 g | Cocotrimonium Methosulfate |
| 0.10 g | Perfume oil/essential oil |
| 3.50 g | Setting polymer or combinations of, e.g., PVP, PVP/VA |
| | Copolymer, Polyquaternium-4, PQ-11, PQ-16, PQ-46, PQ-44, |
| | PQ-68, VP/Methacrylamide/Vinyl Imidazole Copolymer, etc. |
| 0.80 g | Polyetherpolyurethane A.2 |
| q.s. | Preservative |
| 75.00 g | Water dem. |
| 10.00 g | Propane/Butane (3.5 bar) |

Hair styling gel 1
Phase A

| | | |
|---|---|---|
| 0.50 | g | Carbomer or Acrylates/C10-30 Alkyl Acrylate Crosspolymer |
| 86.40 | g | Water dem. |

Phase B

| | | |
|---|---|---|
| 0.70 | g | Triethanolamine |

Phase C

| 11.00 | g | Setting polymer or combinations of, e.g., PVP, PVP/VA |
|---|---|---|
| | | Copolymer, Polyquaternium-4, PQ-11, PQ-16, |
| | | PQ-46, PQ-44, PQ-68, VP/Methacrylamide/Vinyl |
| | | Imidazole Copolymer, etc. |
| 0.20 | g | PEG-25 PABA |
| 2.00 | g | Polyetherpolyurethane A.2 |
| 0.10 | g | Perfume oil/essential oil |
| q.s. | | PEG-14 Dimethicone |
| q.s. | | Preservative |
| 0.10 | g | Tocopheryl Acetate |

Hair styling gel 2
Phase A

| | | |
|---|---|---|
| 0.50 | g | Carbomer or Acrylates/C10-30 Alkyl Acrylate Crosspolymer |
| 91.20 | g | Water dem. |

Phase B

| | | |
|---|---|---|
| 0.90 | g | Tetrahydroxypropyl Ethylenediamine |

Phase C

| 7.00 | g | VP/VA Copolymer |
|---|---|---|
| 0.70 | g | Polyetherpolyurethane A.2 |
| 0.20 | g | Perfume oil/essential oil |
| q.s. | | Preservative |
| 0.10 | g | Propylene Glycol |

Hair wax cream

| 6.00 | g | Caprylic/Capric Triglyceride |
|---|---|---|
| 3.00 | g | Glyceryl Stearate |
| 2.00 | g | Cetyl Alcohol |
| 3.50 | g | Polyetherpolyurethane A.2 |
| 0.50 | g | Cremophor A6 |
| 0.70 | g | Cremophor A25 |
| 0.50 | g | Dimethicone |
| 0.50 | g | Vitamin E Acetate |
| 2.00 | g | Caprylic/Capric Triglyceride and Sodiumacrylates |
| | | Copolymer |
| 1.00 | g | D-Panthenol USP |
| 0.10 | g | EDTA |
| 10.00 | g | Setting polymer |
| q.s. | | Preservative |
| ad 100 | g | Water dem. |

Hair pudding

| 3.00 | g | Kollicoat IR (BASF) |
|---|---|---|
| q.s. | | Preservative |
| 2.00 | g | Setting polymer |
| 4.00 | g | Acrylates/beheneth-25 Methacrylate Copolymer |
| 0.70 | g | Polyetherpolyurethane A.2 |
| 0.50 | g | Dimethicone Copolyol |
| 0.10 | g | EDTA |
| 0.20 | g | Benzophenone-4 |
| ad 100 | g | Water dem. |

Spray gel
Phase A

| | |
|---|---|
| 1.25 g | Setting polymer |
| 96.15 g | Aqua dem. |

Phase B

| | |
|---|---|
| 0.70 g | Acrylates/Steareth-20 Itaconate Copolymer |
| 0.10 g | Propylene Glycol |
| 0.50 g | Polyetherpolyurethane A.2 |
| 0.10 g | Glycerol |
| 0.10 g | Perfume oil/essential oil |
| q.s. | Preservative |

Phase C

| | |
|---|---|
| 0.70 g | Triethanolamine |

A preparation suitable in accordance with the invention for styling sprays may have the following exemplary composition:

Pump hairspray

| | |
|---|---|
| 11.20 g | PEG/PPG-25/25 Dimethicone/Acrylates Copolymer or |
| | Acrylates Copolymer |
| 2.80 g | VP/VA Copolymer |
| 1.34 g | Aminomethyl Propanol |
| 0.30 g | Polyetherpolyurethane A.2 |
| 0.10 g | Perfume oil/essential oil |
| 11.26 g | Aqua dem. |
| 73.00 g | Alcohol |

Pump hairspray VOC55

| | | |
|---|---|---|
| 2.00 | g | VP/Methacrylamide/Vinyl Imidazole Copolymer |
| 1.90 | g | Polyquaternium-46 |
| 2.00 | g | Polyetherpolyurethane A.2 |
| 0.10 | g | Perfume oil/essential oil |
| 55.00 | g | Alcohol |
| 39.00 | g | Aqua dem. |

### Decorative Cosmetic Compositions

Liquid make-up
Phase A

| | | |
|---|---|---|
| 1.70 | g | Glyceryl Stearate |
| 1.70 | g | Cetyl Alcohol |
| 1.70 | g | Ceteareth-6 |
| 1.70 | g | Ceteareth-25 |
| 5.20 | g | Caprylic/Capric Triglyceride |
| 5.20 | g | Mineral Oil or Luvitol ® Lite |
| | | (INCI Hydrogenated Polyisobutene) |

Phase B

| | |
|---|---|
| q.s. | Preservative |

| | | |
|---|---|---|
| 4.30 | g | Propylene Glycol |
| 2.50 | g | Polyetherpolyurethane A.2 |
| 59.50 | g | Aqua dem. |

Phase C

| | | |
|---|---|---|
| 0.10 | g | Perfume oil/essential oil |

Phase D

| | | |
|---|---|---|
| 2.00 | g | Iron Oxides |
| 12.00 | g | Titanium Dioxide |

Eyeliner
Phase A

| | | |
|---|---|---|
| 40.60 | g | Distilled water |
| 0.20 | g | Disodium EDTA |

| | |
|---|---|
| q.s. | Preservative |

Phase B

| | | |
|---|---|---|
| 0.60 | g | Xanthan Gum |
| 0.40 | g | Veegum |
| 3.00 | g | Butylene Glycol |
| 0.20 | g | Polysorbate-20 |

Phase C

| | | |
|---|---|---|
| 15.00 | g | Iron oxide/Al Powder/Silica (e.g., |
| | | Sicopearl ® Fantastico Gold from |
| | | BASF, or other effect pigments) |

Phase D

| | | |
|---|---|---|
| 10.00 | g | Aqua dem. |
| 25.00 | g | Setting polymer or combinations |
| | | of, e.g., PVP, PVP/VA Copolymer, |
| | | Polyquaternium-4, PQ-11, PQ-16, |
| | | PQ-46, PQ-44, PQ-68, Polyurethane-1 or |
| | | VP/Methacrylamide/ Vinyl Imidazole |
| | | Copolymer, etc. |
| 5.00 | g | Polyetherpolyurethane A.2 |

Face lotion
Phase A

| | | |
|---|---|---|
| 3.00 | g | Polyetherpolyurethane A.2 |
| 0.10 | g | Perfume oil/essential oil |
| 0.30 | g | Bisabolol |

Phase B

| | | |
|---|---|---|
| 3.00 | g | Glycerol |
| 1.00 | g | Hydroxyethyl Cetyldimonium Phosphate |
| 5.00 | g | Witch Hazel (Hamamelis Virginiana) |
| | | Distillate |
| 0.50 | g | Panthenol |

| | |
|---|---|
| q.s. | Preservative |

| | | |
|---|---|---|
| 87.60 | g | Aqua dem. |

Face wash paste with peeling effect
Phase A

| | | |
|---|---|---|
| 73.00 | g | Aqua dem. |
| 1.50 | g | Polyetherpolyurethane A.2 |

| | |
|---|---|
| q.s. | Preservative |

Phase B

| | |
|---|---|
| q.s. | Perfume oil |

| | | |
|---|---|---|
| 7.00 | g | Potassium Cocoyl Hydrolyzed Protein |
| 4.00 | g | Conditioning polymer or combinations of |
| | | Polyquaternium-7, PQ-10, PQ-39, PQ-44, |
| | | PQ-67, Guarhydroxypropyltrimonium |
| | | Chloride, PQ-87 |

Phase C

| | | |
|---|---|---|
| 1.50 | g | Triethanolamine |

Phase D

| | | |
|---|---|---|
| 13.00 | g | Polyethylene (Luwax A ™ from BASF) |

Soap
Phase A

| | | |
|---|---|---|
| 25.00 | g | Potassium Cocoate |
| 20.00 | g | Disodium Cocoamphodiacetate |
| 2.00 | g | Lauramide DEA |
| 1.0 | g | Glycol Stearate |
| 2.00 | g | Polyetherpolyurethane A.2 |
| 0.50 | g | Conditioning polymer or combinations of |
| | | Polyquaternium-7, PQ-10, PQ-39, PQ-44, |
| | | PQ-67, Guarhydroxypropyltrimonium |
| | | Chloride, PQ-87 |
| 50.00 | g | Aqua dem. |

| | |
|---|---|
| q.s. | Citric Acid |

Phase B

| | |
|---|---|
| q.s. | Preservative |

| | | |
|---|---|---|
| 0.10 | g | Perfume oil/essential oil |

Face cleansing milk, O/W type
Phase A

| | | |
|---|---|---|
| 1.50 | g | Ceteareth-6 |
| 1.50 | g | Ceteareth-25 |
| 2.00 | g | Glyceryl Stearate |
| 2.00 | g | Cetyl Alcohol |
| 10.00 | g | Mineral Oil |

Phase B

| | | |
|---|---|---|
| 5.00 | g | Propylene Glycol |

| | |
|---|---|
| q.s. | Preservative |

| | | |
|---|---|---|
| 1.00 | g | Conditioning polymer or combinations of |
| | | Polyquaternium-7, PQ-10, PQ-39, PQ-44, |
| | | PQ-67, Guarhydroxypropyltrimonium |
| | | Chloride, PQ-87 |
| 66.30 | g | Aqua dem. |

Phase C

| | | |
|---|---|---|
| 0.20 | g | Polyetherpolyurethane A.2 |
| 10.00 | g | Cetearyl Octanoate |

Phase D

| | | |
|---|---|---|
| 0.40 | g | Tetrahydroxypropyl Ethylenediamine |

Phase E

| | | |
|---|---|---|
| 0.10 | g | Perfume oil/essential oil |
| 0.10 | g | Bisabolol |

Transparent soap

| | | |
|---|---|---|
| 4.20 | g | Sodium Hydroxide |
| 3.60 | g | Distilled water |
| 5.00 | g | Conditioning polymer or combinations of |
| | | Polyquaternium-7, PQ-10, PQ-39, PQ-44, |
| | | PQ-67, Guarhydroxypropyltrimonium |
| | | Chloride, PQ-87 |
| 5.00 | g | Polyetherpolyurethane A.2 |
| 22.60 | g | Propylene Glycol |
| 18.70 | g | Glycerol |
| 5.20 | g | Cocoamide DEA |
| 2.40 | g | Cocamine Oxide |
| 4.20 | g | Sodium Lauryl Sulfate |
| 7.30 | g | Myristic Acid |
| 16.60 | g | Stearic Acid |
| 5.20 | g | Tocopherol |

Shaving foam

| | | |
|---|---|---|
| 6.00 | g | Ceteareth-25 |
| 5.00 | g | Poloxamer 407 |
| 52.00 | g | Aqua dem. |
| 1.00 | g | Triethanolamine |
| 5.00 | g | Propylene Glycol |
| 1.00 | g | PEG-75 Lanolin Oil |
| 2.00 | g | Conditioning polymer or combinations of |
| | | Polyquaternium-7, PQ-10, PQ-39, PQ-44, |
| | | PQ-67, Guarhydroxypropyltrimonium |
| | | Chloride, PQ-87 |
| 3.00 | g | Polyetherpolyurethane A.2 |

| | |
|---|---|
| q.s. | Preservative |

| | | |
|---|---|---|
| 0.10 | g | Perfume oil/essential oil |
| 25.00 | g | Sodium Laureth Sulfate |

Filling: 90 parts of active substance and 10 parts of 25:75 propane/butane mixture.

After Shave Balsam
Phase A

| | | |
|---|---|---|
| 0.25 | g | Polyetherpolyurethane A.2 |
| 1.50 | g | Tocopheryl Acetate |
| 0.20 | g | Bisabolol |
| 10.00 | g | Caprylic/Capric Triglyceride |

| | |
|---|---|
| q.s. | Perfume |

| | | |
|---|---|---|
| 1.00 | g | Conditioning polymer or combinations of |
| | | Polyquaternium-7, PQ-10, PQ-39, PQ-44, |
| | | PQ-67, Guarhydroxypropyltrimonium |
| | | Chloride, PQ-87 |

Phase B

| | | |
|---|---|---|
| 1.00 | g | Panthenol |
| 15.00 | g | Alcohol |
| 5.00 | g | Glycerol |
| 0.05 | g | Hydroxyethyl Cellulose |
| 1.90 | g | Polyetherpolyurethane A.2 |
| 64.02 | g | Distilled water |

Phase C

| | | |
|---|---|---|
| 0.08 | g | Sodium Hydroxide |

Toothpaste
Phase A

| | | |
|---|---|---|
| 34.79 | g | Aqua dem. |
| 3.00 | g | Polyetherpolyurethane A.2 |
| 20.00 | g | Glycerol |
| 0.76 | g | Sodium Monofluorophosphate |

Phase B

| | | |
|---|---|---|
| 1.20 | g | Sodium Carboxymethylcellulose |

Phase C

| | | |
|---|---|---|
| 0.80 | g | Aroma oil |
| 0.06 | g | Saccharin |

| | |
|---|---|
| q.s. | Preservative |

| | | |
|---|---|---|
| 0.05 | g | Bisabolol |
| 1.00 | g | Panthenol |
| 0.50 | g | Tocopheryl Acetate |
| 2.80 | g | Silica |
| 1.00 | g | Sodium Lauryl Sulfate |
| 7.90 | g | Dicalciumphosphate Anhydrate |
| 25.29 | g | Dicalciumphosphate Dihydrate |
| 0.45 | g | Titanium Dioxide |

Mouthwash
Phase A

| | | |
|---|---|---|
| 2.00 | g | Aroma oil |
| 4.50 | g | Polyetherpolyurethane A.2 |
| 1.00 | g | Bisabolol |
| 30.00 | g | Alcohol |

Phase B

| | | |
|---|---|---|
| 0.20 | g | Saccharin |
| 5.00 | g | Glycerol |

| | |
|---|---|
| q.s. | Preservative |

| | | |
|---|---|---|
| 5.00 | g | Poloxamer 407 |
| 52.30 | g | Aqua dem. |

Prosthesis adhesive
Phase A

| | | |
|---|---|---|
| 0.20 | g | Bisabolol |
| 1.00 | g | Beta-Carotene |

| | |
|---|---|
| q.s. | Aroma oil |

| | | |
|---|---|---|
| 20.00 | g | Cetearyl Octanoate |
| 5.00 | g | Silica |
| 33.80 | g | Mineral Oil |

Phase B

| | | |
|---|---|---|
| 5.00 | g | Polyetherpolyurethane A.2 |
| 35.00 | g | PVP (20% strength solution in water) |

Self-tanning cream
Phase A

| | | |
|---|---|---|
| 2.00 | Jeechem ® BUGL | Butylene Glycol |
| 3.00 | Polyetherpoly- | |
| | urethane A.2 | |
| ad 100 | Water dem. | Aqua dem. |

Phase B

| | | |
|---|---|---|
| 1.00 | Lipovol ® J | Simmondsia Chinensis (Jojoba) Seed |
| | | Oil |
| 0.75 | Span ® 60 | Sorbitan Stearate |
| 2.50 | Lipomulse ® 165 | Glyceryl Stearate, PEG-100 Stearate |
| 2.00 | Luvitol ® Lite | Hydrogenated Polyisobutene |
| 1.00 | Dow Corning ® 200 | Dimethicone |
| | Fluid | |
| 2.50 | Crodacol CS-50 | Cetearyl Alcohol |
| 1.75 | Cremophor ® A 6 | Ceteareth-6, Stearyl Alcohol |
| 0.50 | Vitamin E Acetate | Tocopheryl Acetate |
| | Care | |
| 0.50 | Bisabolol rac. | Bisabolol |
| 1.00 | Phenonip | Phenoxyethanol, Methylparaben, |
| | | Ethylparaben, Butylparaben, |
| | | Propylparaben, Isobutylparaben |

Phase C

| | | |
|---|---|---|
| 10.00 | Water dem. | Aqua dem. |
| 5.00 | Dihydroxyacetone | Dihydroxyacetone |

Phase D

| | | |
|---|---|---|
| 0.04 | Citric acid | Citric Acid |
| q.s. | Perfume oil | |

Self-tanning lotion

| | |
|---|---|
| Ingredient | INCI |

Phase A

| | | |
|---|---|---|
| 4.00 | Luvitol ® Lite | Hydrogenated Polyisobutene |
| 2.00 | Lanette ® O | Cetearyl Alcohol |
| 1.00 | Isopropyl palmitate | Isopropyl Palmitate |
| 1.00 | Eutanol ® G | Octyldodecanol |
| 1.00 | Jojoba oil | Simmondsia Chinensis (Jojoba) Seed |
| | | Oil |
| 1.00 | Abil ® 350 | Dimethicone |
| 1.00 | Amphisol ® K | Potassium Cetyl Phosphate |
| 0.50 | Emulmetik ® 100 | Lecithin |
| 0.50 | Tego Care ® CG 90 | Cetearyl Glucoside |

Phase B

| | | |
|---|---|---|
| 1.00 | Euxyl ® K 320 | Propylene Glycol, Phenoxyethanol, |
| | | Methylparaben, Ethylparaben |
| 4.00 | Glycerol | Glycerin |
| 1.00 | Polyetherpoly- | A.2 |
| | urethane | |
| 0.10 | Keltrol | Xanthan Gum |
| ad 100 | Water dem. | Aqua dem. |

Phase C

| | | |
|---|---|---|
| 1.50 | Dihydroxyacetone | Dihydroxyacetone |
| 10.00 | Water dem. | Aqua dem. |
| q.s. | Perfume oil | |

### Preparation

Heat phases A and B separately to approximately 80° C.

Stir phase B into phase A and briefly homogenize.

Cool to approximately 40° C. with stirring, add phase C, and briefly homogenize again.

What is claimed is:

1. A polymer compound comprising at least one end group of the general formula (I) 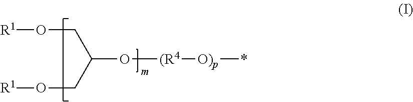
wherein:
R¹ is independently at each occurrence C₄-C₄₀-alkyl, C₃-C₁₀-cycloalkyl, C₆-C₃₀-aryl, C₇-C₄₀-aralkyl, C₇-C₄₀-alkylaryl or —(R²—O)ₙ—R³;
R², R⁴ are independently at each occurrence C₂-C₁₀-alkylene, C₆-C₁₀-arylene, or C₇-C₁₀-aralkylene;
R³ is H, C₁-C₄₀-alkyl, C₃-C₁₀-cycloalkyl, C₆-C₃₀-aryl, C₇-C₄₀-aralkyl or C₇-C₄₀-alkylaryl;
n is 1 to 200;
m is 1 to 6;
p is 0 to 200;
* represents linkage of the at least one end group to the polymer compound;
with the proviso that, when p=0, the at least one end group, rather than via O, may also be linked to the rest of the compound via NR⁵, wherein R⁵ is selected from H and C₁-C₃₀-alkyl, and
wherein * comprises a structural unit selected from esters, amides, imides, urethanes, ureas, amines, ethers, or carbonates.

2. The polymer compound according to claim 1, wherein R¹ is —(R²—O)ₙ—R³, R² is C₂-C₅-alkylene, R⁴ is C₂-C₅-alkylene, R³ is C₁₂-C₃₀-alkyl or C₇-C₃₀-aralkyl, and p=0.

3. The polymer compound according to claim 1, wherein m is in the range from 2 to 5.

4. The polymer compound according to claim 1, wherein n is in the range from 4 to 50.

5. The polymer compound according to claim 1, wherein p is in the range from 0 to 5.

6. The polymer compound according to claim 1, wherein * comprises a structural unit selected from urethanes, ureas, esters, amides, and carbonates.

7. The polymer compound according to claim 6, wherein the polymer compound is a polyetherurethane and comprises each in copolymerized from
a. at least one polyether polyol and
b. at least one polyisocyanate.

8. The polymer compound according to claim 7, wherein the at least one polyether polyol has a number-average molecular weight Mn in the range from 4,000 to 10,000 g/mol.

9. The polymer compound according to claim 8, wherein the at least one polyether polyol has a number-average molecular weight Mn in the range from 6,000 to 9,000 g/mol.

10. The polymer compound according to claim 6, wherein the polymer compound is a polyetherurethane and comprises each in copolymerized form
a. at least one polyester polyol and
b. at least one polyisocyanate.

11. The polymer compound according to claim 10, wherein the at least one polyester polyol has a number-average molecular weight Mn in the range from 400 to 5,000 g/mol.

12. The polymer compound according to claim 11, wherein the at least one polyester polyol has a number-average molecular weight Mn in the range from 400 to 2,000 g/mol.

13. The polymer compound according to claim 1, wherein * comprises a structural unit selected from urethanes and ureas.

14. An aqueous preparation comprising at least one polymer compound according to claim 1.

15. The aqueous preparation according to claim 14, further comprising at least one salt or surfactant or both.

16. An aqueous preparation comprising the at least one polymer compound according to claim 13.

17. The aqueous preparation according to claim 16, further comprising at least one salt or surfactant or both.
